SEQUENCE LISTING (42 sequences)

SEQ ID NO: 1 - DNA, 34 bp, Artificial Sequence
  Synthetic
gctgaggggtgcttccagcgaggcggcctcttcc

SEQ ID NO: 2 - DNA, 33 bp, Artificial Sequence
  Synthetic
aggcctccagctcccccggggcctccgtttcta

SEQ ID NO: 3 - DNA, 32 bp, Artificial Sequence
  Synthetic
tgcagaaactccgggctcgccaggagctcatc

SEQ ID NO: 4 - DNA, 44 bp, Artificial Sequence
  Synthetic
aaccccgcgtcctaaagctcctccagcagagcccggtattcttc

SEQ ID NO: 5 - DNA, 48 bp, Artificial Sequence
  Synthetic
gctcctccagcagagcccggtattcttcctcgctgaggggtgcttcca

SEQ ID NO: 6 - DNA, 35 bp, Artificial Sequence
  Synthetic
cgaaccacccgaccccgtcccaaccccgcgtccta

SEQ ID NO: 7 - DNA, 32 bp, Artificial Sequence
  Synthetic
agctcctccagcagagcccggtattcttcctc

SEQ ID NO: 8 - DNA, 30 bp, Artificial Sequence
  Synthetic
ggagggggcattttaatatatctctgaact

SEQ ID NO: 9 - DNA, 29 bp, Artificial Sequence
  Synthetic
gtgctgtccgagggtgtcgggagggccat

SEQ ID NO: 10 - DNA, 45 bp, Artificial Sequence
  Synthetic
ggcttccgcggggagggtgctgtccgagggtgtcgggagggccat

SEQ ID NO: 11 - DNA, 47 bp, Artificial Sequence
  Synthetic
gaaccacccgaccccgtcccaaccccgcgtcctaaagctcctccagc

SEQ ID NO: 12 - DNA, 64 bp, Artificial Sequence
  Synthetic
gaaccacccgaccccgtcccaaccccgcgtcctaaagctcctccagcagagcccggtattcttc

SEQ ID NO: 13 - DNA, 32 bp, Artificial Sequence
  Synthetic
gtgcgcagtaggcggcccacctgctggtacct

SEQ ID NO: 14 - DNA, 35 bp, Artificial Sequence
  Synthetic
cgcgcaggtctagtcaggaagcgggcaaagacaga

SEQ ID NO: 15 - DNA, 65 bp, Artificial Sequence
  Synthetic
cggggtgcgcactgcgcgcaggtctagtcaggaagcgggcaaagacagacagaggtatgcttttg

SEQ ID NO: 16 - DNA, 42 bp, Artificial Sequence
  Synthetic
gcacgtcagccggggtgcgcactgcgcgcaggtctagtcagg

SEQ ID NO: 17 - DNA, 40 bp, Artificial Sequence
  Synthetic
gggggcattttaatatatctctgaactaatcatccaggag

SEQ ID NO: 18 - DNA, 61 bp, Artificial Sequence
  Synthetic
ggagggggcattttaatatatctctgaactaatcatccaggagatgtaactctaatccagg

SEQ ID NO: 19 - DNA, 34 bp, Artificial Sequence
  Synthetic
ggaagaggccgcctcgctggaagcacccctcagc

SEQ ID NO: 20 - DNA, 33 bp, Artificial Sequence
  Synthetic
tagaaacggaggccccgggggagctggaggcct

SEQ ID NO: 21 - DNA, 32 bp, Artificial Sequence
  Synthetic
gatgagctcctggcgagcccggagtttctgca

SEQ ID NO: 22 - DNA, 44 bp, Artificial Sequence
  Synthetic
gaagaataccgggctctgctggaggagctttaggacgcggggtt

SEQ ID NO: 23 - DNA, 48 bp, Artificial Sequence
  Synthetic
tggaagcacccctcagcgaggaagaataccgggctctgctggaggagc

SEQ ID NO: 24 - DNA, 35 bp, Artificial Sequence
  Synthetic
taggacgcggggttgggacggggtcgggtggttcg

SEQ ID NO: 25 - DNA, 32 bp, Artificial Sequence
  Synthetic
gaggaagaataccgggctctgctggaggagct

SEQ ID NO: 26 - DNA, 30 bp, Artificial Sequence
  Synthetic
agttcagagatatattaaaatgccccctcc

SEQ ID NO: 27 - DNA, 29 bp, Artificial Sequence
  Synthetic
atggccctcccgacaccctcggacagcac

SEQ ID NO: 28 - DNA, 45 bp, Artificial Sequence
  Synthetic
atggccctcccgacaccctcggacagcaccctccccgcggaagcc

SEQ ID NO: 29 - DNA, 47 bp, Artificial Sequence
  Synthetic
gctggaggagctttaggacgcggggttgggacggggtcgggtggttc

SEQ ID NO: 30 - DNA, 64 bp, Artificial Sequence
  Synthetic
gaagaataccgggctctgctggaggagctttaggacgcggggttgggacggggtcgggtggttc

SEQ ID NO: 31 - DNA, 32 bp, Artificial Sequence
  Synthetic
aggtaccagcaggtgggccgcctactgcgcac

SEQ ID NO: 32 - DNA, 35 bp, Artificial Sequence
  Synthetic
tctgtctttgcccgcttcctgactagacctgcgcg

SEQ ID NO: 33 - DNA, 65 bp, Artificial Sequence
  Synthetic
caaaagcatacctctgtctgtctttgcccgcttcctgactagacctgcgcgcagtgcgcaccccg

SEQ ID NO: 34 - DNA, 42 bp, Artificial Sequence
  Synthetic
cctgactagacctgcgcgcagtgcgcaccccggctgacgtgc

SEQ ID NO: 35 - DNA, 40 bp, Artificial Sequence
  Synthetic
ctcctggatgattagttcagagatatattaaaatgccccc

SEQ ID NO: 36 - DNA, 61 bp, Artificial Sequence
  Synthetic
cctggattagagttacatctcctggatgattagttcagagatatattaaaatgccccctcc

SEQ ID NO: 37 - DNA, 1275 bp, Artificial Sequence
  Synthetic
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttag

SEQ ID NO: 38 - protein, 424 aa, Artificial Seuqence
  Synthetic
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 39 - DNA, 61 bp, Artificial Sequence
  Synthetic
gaatcgagcaccagttacgcatgccgaggtcgacttcctagattttttttttttttttttt

SEQ ID NO: 40 - DNA, 21 bp, Artificial Sequence
  Synthetic
cagaatgagaggtcacgccag

SEQ ID NO: 41 - DNA, 23 bp, Artificial Sequence
  Synthetic
gaatcgagcaccagttacgcatg

SEQ ID NO: 42 - DNA, 20 bp, Artificial Sequence
  Synthetic
gagcaccagttacgcatgcc

(19) United States
(12) Patent Application Publication
(10) Pub. No.: US 2024/0026356 A1
(43) Pub. Date: Jan. 25, 2024

(54) COMPOSITIONS AND METHODS FOR TREATING FACIOSCAPULOHUMERAL MUSCULAR DYSTROPHY (FSHD)

(71) Applicant: RESEARCH INSTITUTE AT NATIONWIDE CHILDREN'S HOSPITAL, Columbus, OH (US)

(72) Inventors: Scott Quenton Harper, Powell, OH (US); Afrooz Rashnonejad, Columbus, OH (US); Nicolas Sebastien Wein, Columbus, OH (US)

(21) Appl. No.: 18/038,878

(22) PCT Filed: Nov. 30, 2021
(86) PCT No.: PCT/US21/61109

§ 371 Date: May 25, 2023

Related U.S. Application Data
(60) Provisional application No. 63/119,268, filed on Nov. 30, 2020.

(51) Int. Cl. C12N 15/113; C12N 15/86; A61P 21/00

(52) U.S. Cl. CPC C12N 15/113; C12N 15/86; A61P 21/00; C12N 2310/11; C12N 2750/14143

(57) ABSTRACT

Disclosed herein are products, methods, and uses for treating, ameliorating, udaying the progression of, and/or preventing a muscular dystrophy or a cancer including, but not limited to, facioscapulohumeral muscular dystrophy (FSHD) or a sarcoma. More particularly, disclosed herein are RNA interference-based products, methods, and uses for inhibiting or downregulating the expression of double homeobox 4 (DUX4). Even more particularly, the disclosure provides nucleic acids comprising U7 DUX4 antisense sequences for inhibiting or downregulating the expression of DUX4 and methods of using said antisense sequences to inhibit or downregulate DUX4 expression in cells and/or in cells of a subject having a muscular dystrophy or a cancer including, but not limited to, FSHD or a cancer.

### STATEMENT OF GOVERNMENT INTEREST

This invention was made with government support under grant no. AR070604 from the National Institutes of Health. The government has certain rights in the invention.

### INCORPORATION BY REFERENCE OF THE SEQUENCE LISTING

This application contains, as a separate part of disclosure, a Sequence Listing in computer-readable form (Filename: 56061_Seqlisting.txt; Size: 13,671 bytes; Created: Nov. 23, 2021) which is incorporated by reference herein in its entirety.

### FIELD

This disclosure relates to the field of the treatment of a muscular dystrophy or a cancer including, but not limited to, facioscapulohumeral muscular dystrophy (FSHD) or a sarcoma. More particularly, the disclosure provides RNA interference-based products, methods, and uses for treating, ameliorating, delaying the progression of, and/or preventing a muscular dystrophy or a cancer including, but not limited to, FSHD or a sarcoma. Specifically, the disclosure provides products and methods for inhibiting or downregulating the expression of the double homeobox 4 (DUX4) gene. More specifically, the disclosure provides U7 small nuclear RNA (U7 snRNA) for inhibiting or downregulating the expression of DUX4 and methods of using said U7 snRNA to inhibit or downregulate DUX4 expression in cells and/or in a subject having or at risk of having a muscular dystrophy or a cancer.

### BACKGROUND

Muscular dystrophies (MDs) are a group of genetic diseases. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

Facioscapulohumeral dystrophy (FSHD) is among the most commonly inherited muscular dystrophies, estimated to affect as many as 870,000 individuals. Classical descriptions of FSHD presentation include progressive muscle weakness in the face, shoulder-girdle and arms, but disease can manifest more broadly, including in muscles of the trunk and lower extremities. Variability is also commonly seen within individuals, as asymmetrical weakness is common. Age-at-onset can range from early childhood to adulthood, and is usually related to disease severity, where earlier onset is often associated with more severe muscle weakness. Although most patients with FSHD have a normal life span, respiratory insufficiency can occur, and the disease can be debilitating, as approximately 25% of affected individuals may become wheelchair dependent by their fifties, and even earlier in more severe forms of the disease, while others maintain lifelong ambulation.

FSHD is caused by aberrant expression of the double homeobox 4 gene (DUX4), which produces a transcription factor that is toxic to skeletal muscle. DUX4 is normally functional during the four-cell stage of human development but repressed thereafter in essentially all other tissues, except perhaps the testes and possibly the thymus. In skeletal muscles of people with FSHD, specific genetic and epigenetic factors conspire to permit DUX4 de-repression, where it then initiates several aberrant gene expression cascades, including those involved in differentiation abnormalities, oxidative stress, inflammatory infiltration, cell death and muscle atrophy.

Despite progress in the FSHD field, there are still no approved treatments for FSHD, and therapeutic development remains a critical need in the field. The safety and efficacy of DUX4 silencing using RNAi-based gene therapy delivered by AAV vectors in pre-clinical studies has been shown previously (Wallace et al., Mol Ther Methods Clin Dev 8, 121-130 (2018)). Because even very small amounts of DUX4 protein may be toxic in muscle cells, it is crucial to develop additional DUX4 silencing strategies employing alternative mechanisms, which could be used alone or in combinatorial therapies, to help maximize DUX4 silencing in patient muscles.

Since FSHD arises from DUX4 de-repression, the most direct route to a therapy will involve inhibiting DUX4 in muscle. Gene regulation by U7 small nuclear RNA (U7 snRNA) is one powerful approach to inhibit DUX4. U7 snRNA is an RNA molecule and a component of the small nuclear ribonucleoprotein complex (U7 snRNP) and is required for histone pre-mRNA processing.

Viral vectors, such as adeno-associated virus (AAV), have been used to deliver U7 snRNAs to muscle. AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hardy virus. It easily withstands the conditions used to inactivate adenovirus (56° to 65° C. for several hours), making cold preservation of AAV less critical. AAV may even be lyophilized. Finally, AAV-infected cells are not resistant to superinfection.

There remains a need in the art for products and methods for treating muscular dystrophies including, but not limited to, FSHD. The disclosure provides a U7 small nuclear RNA (U7 snRNA) approach to inhibit DUX4 expression in muscle cells.

### SUMMARY

The disclosure provides products, methods, and uses for inhibiting DUX4 expression and for treating, ameliorating, delaying the progression of, and/or preventing a muscular dystrophy. In some aspects, the muscular dystrophy is facioscapulohumeral dystrophy (FSHD).

The disclosure provides nucleic acids, viral vectors comprising the nucleic acids which are designed to inhibit DUX4 expression, compositions and kits comprising the nucleic acids and vectors, methods for using these products for inhibiting and/or interfering with expression of a DUX4 gene in a cell, and methods for treating a subject suffering from a muscular dystrophy.

The disclosure provides a nucleic acid encoding a U7 double homeobox 4 (DUX4) antisense ribonucleic acid (asRNA), the nucleic acid comprising (a) a nucleotide sequence comprising at least 90% identity to the sequence set forth in any one of SEQ ID NOs: 1-18; (b) the nucleotide sequence set forth in any one of SEQ ID NOs: 1-18; or (c) a combination of the nucleotide sequences of (a) and/or (b).

The disclosure provides a nucleic acid comprising a nucleotide sequence encoding a U7 double homeobox 4 (DUX4) antisense sequence that specifically hybridizes to a DUX4 target nucleotide sequence set forth in any one of SEQ ID NOs: 19-36, or a combination of nucleotide sequences encoding a U7 double homeobox 4 (DUX4) antisense sequence that specifically hybridizes to a DUX4 target nucleotide sequence set forth in any one of SEQ ID NOs: 19-36.

In some aspects, a nucleic acid of the disclosure is under the control of a promoter and therefore comprises a promoter nucleotide sequence. In some aspects, the promoter is any of a U6 promoter, a U7 promoter, a tRNA promoter, a H1 promoter, a minimal CMV promoter, a T7 promoter, an EF1-alpha promoter, a Minimal EF1-alpha promoter, or a muscle-specific promoter. In some further aspects, the muscle-specific promoter is wherein the muscle-specific promoter is a unc45b promoter, a tMCK promoter, a minimal MCK promoter, a CK6 promoter, a CK7 promoter, a MHCK7 promoter, or a CK1 promoter.

In some aspects, the disclosure includes a nanoparticle, extracellular vesicle, exosome, or vector comprising any of the nucleic acids of the disclosure or a combination of any one or more thereof. In some aspects, one or more nucleic acids are combined into a single nanoparticle, extracellular vesicle, exosome, or vector. In some aspects, the nanoparticle is a liposome or micelle.

In some aspects, the disclosure includes a vector comprising a nucleic acid of the disclosure or a combination of nucleic acids of the disclosure. Embodiments of the disclosure utilize vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein. Thus, in some aspects, the disclosure provides a vector comprising any one of the nucleic acids of the disclosure or a combination thereof. In some aspects, the vector is an adeno-associated virus (AAV) or viral vector. In some aspects, the AAV lacks rep and cap genes. In some aspects, the AAV is a recombinant AAV (rAAV) or a self-complementary recombinant AAV (scAAV). In some aspects, the rAAV is rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh.10, or rAAV-B1. In some aspects, the AAV is rAAV-9.

The disclosure provides a composition comprising (a) a nucleic acid as described herein or a combination of such nucleic acids; (b) a nanoparticle, extracellular vesicle, exosome, or vector as described herein; (c) a viral vector as described herein; or (d) a composition as described herein. In some aspects, the composition comprises a pharmaceutically acceptable carrier.

The disclosure provides a method of inhibiting and/or interfering with expression of a double homeobox 4 (DUX4) gene in a cell comprising contacting the cell with (a) a nucleic acid of the disclosure or a combination thereof; (b) a nanoparticle, extracellular vesicle, exosome, or vector of the disclosure; (c) a viral vector of the disclosure; or (d) a composition of the disclosure. In some aspects, the cell is in a subject. In some aspects, the subject is a human subject.

The disclosure provides a method of treating a subject having a muscular dystrophy comprising administering to the subject an effective amount of (a) a nucleic acid of the disclosure or a combination thereof; (b) a nanoparticle, extracellular vesicle, exosome, or vector of the disclosure; (c) a viral vector of the disclosure; or (d) a composition of the disclosure. In some aspects, the muscular dystrophy is facioscapulohumeral muscular dystrophy (FSHD).

The disclosure provides a method of treating a subject having a cancer comprising administering to the subject an effective amount of (a) a nucleic acid of the disclosure or a combination thereof; (b) a nanoparticle, extracellular vesicle, exosome, or vector of the disclosure; (c) a viral vector of the disclosure; or (d) a composition of the disclosure. In some aspects, the cancer is a sarcoma.

The disclosure provides use of (a) a nucleic acid of the disclosure or a combination thereof; (b) a nanoparticle, extracellular vesicle, exosome, or vector of the disclosure; (c) a viral vector of the disclosure; or (d) a composition of the disclosure for the preparation of a medicament for inhibiting expression of a double homeobox 4 (DUX4) gene in a cell. In some aspects, the cell is in a subject. In some aspects, the subject is a human subject.

The disclosure provides use of (a) a nucleic acid of the disclosure or a combination thereof; (b) a nanoparticle, extracellular vesicle, exosome, or vector of the disclosure; (c) a viral vector of the disclosure; or (d) a composition of the disclosure for inhibiting expression of a double homeobox 4 (DUX4) gene in a cell. In some aspects, the cell is in a subject. In some aspects, the subject is a human subject.

The disclosure provides use of (a) a nucleic acid of the disclosure or a combination thereof; (b) a nanoparticle, extracellular vesicle, exosome, or vector of the disclosure; (c) a viral vector of the disclosure; or (d) a composition of the disclosure for the preparation of a medicament for treating or ameliorating a muscular dystrophy.

The disclosure provides use of (a) a nucleic acid of the disclosure or a combination thereof; (b) a nanoparticle, extracellular vesicle, exosome, or vector of the disclosure; (c) a viral vector of the disclosure; or (d) a composition of the disclosure for treating or ameliorating a muscular dystrophy. In some aspects, the muscular dystrophy is FSHD.

The disclosure provides use of (a) a nucleic acid of the disclosure or a combination thereof; (b) a nanoparticle, extracellular vesicle, exosome, or vector of the disclosure; (c) a viral vector of the disclosure; or (d) a composition of the disclosure for the preparation of a medicament for treating or ameliorating a cancer. In some aspects, the cancer is a sarcoma.

The disclosure provides use of (a) a nucleic acid of the disclosure or a combination thereof; (b) a nanoparticle, extracellular vesicle, exosome, or vector of the disclosure; (c) a viral vector of the disclosure; or (d) a composition of the disclosure for treating or ameliorating a cancer. In some aspects, the cancer is a sarcoma.

The disclosure also provides methods and uses, wherein the nucleic acid, nanoparticle, extracellular vesicle, exosome, vector, viral vector, composition, or medicament of the disclosure is formulated for intramuscular injection, transdermal transport or injection into the blood stream.

Further aspects and advantages of the disclosure will be apparent to those of ordinary skill in the art from a review of the following detailed description, taken in conjunction with the drawings. It should be understood, however, that the detailed description (including the drawings and the specific examples), while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWINGS

FIG. 1A-D shows that U7-asDUX4 snRNAs protect HEK293 cells from DUX4-mediated death. FIG. 1A shows U7-snRNA structure consisting of a stabilizing hairpin-loop, Sm binding region, and an antisense sequence complementary to a target site on the DUX4 pre-mRNA (see Table 1 for sequences). FIG. 1B is a schematic drawing of 18 U7-asDUX4 constructs targeting different parts of DUX4 mRNA. ATG indicates start codon. Exon 1 (Ex1) contains the entire DUX4 open reading frame, while Ex2 and Ex3 contain 3′ untranslated regions (3′ UTR). *P≤0.05, ** P≤0.01, ANOVA. N=3 independent experiments performed in triplicate. FIG. 10 shows results of a Caspase-3/7 assay for apoptosis. All DUX4-targeting U7-asDUX4 snRNA constructs significantly reduced Caspase-3/7 activity except in cells treated with sequence 6. Fourteen of 18 constructs tested reduced Caspase-3/7 activity more than 50% (exceptions were 1, 2, 6, and 18). Cells with the lowest relative Caspase-3/7 activity (normalized to activity in cells transfected with DUX4 only) were treated with U7-asDUX4-3 (34±12), U7-asDUX4-4 (26±7), U7-asDUX4-7 (36±8), U7-asDUX4-11 (30±3), and U7-asDUX4-16 (33±6). FIG. 1D shows cell viability increased significantly in all U7-asDUX4-treated cells compared to those transfected with DUX4 alone, with U7-asDUX4-7 (94.8%±4.9), U7-asDUX4-8 (84.7%±3.8), and U7-asDUX4-4 (78.5%±8.4) respectively, showing the most percentage of viable cells (P<0.01, ANOVA; N=3 independent experiments).

FIG. 2A-H shows U7-asDUX4 snRNAs significantly reduced overexpressed DUX4 mRNA in transfected HEK293 cells. RNAscope assay: DUX4 mRNA signals appeared as brown, punctate dots in transfected cells (FIG. 2A-D). FIG. 2A shows abundant DUX4 signal detected in HEK293 cells co-transfected with CMV.DUX4 and a DUX4 non-targeting U7 control plasmid. Reduction in brown DUX4 signal after co-transfection of HEK293 cells with CMV.DUX4 and U7-asDUX4-4 (FIG. 2B), U7-asDUX4-7 (FIG. 2C), and U7-asDUX4-8 (FIG. 2D) plasmids. FIG. 2E shows background signal with DUX4 probe in untransfected HEK293 cell line. FIG. 2F shows housekeeping gene PPIB was detected in all HEK293 cells and served as a positive control for the assay. FIG. 2G shows bacterial gene dapB probe was used a negative control for RNAscope assay. FIG. 2H shows RNAscope quantification showed significantly reduced DUX4-positive signal in DUX4-transfected cells co-expressing U7-asDUX4 snRNAs 4, 7 and 8. 40× objective. Scale bar, 50 microns. Quantification was performed as described in Ref 30 (see citation at end of disclosure). Two representative microscopic fields were counted from 3 independent experiments; each point represents quantification of one field. **P<0.01, ANOVA.

FIG. 3A-E shows that U7-asDUX4 snRNAs reduce DUX4 protein production in transfected HEK293 cells. FIG. 3A shows a schematic of full-length DUX4 expression construct containing a C-terminal V5 epitope tag. The 42 bp DNA sequence encoding the 14 amino acid V5 tag disrupted the U7-asDUX4-4 target site. Black bars in exon 1 (Ex1) indicate DNA binding homeodomains 1 and 2 (HOX1 and HOX2) but are not to scale. Introns 1 and 2 are indicated as ∨ symbols. FIG. 3B shows anti-V5 immunofluorescence staining of HEK293 cells co-transfected with CMV.DUX4-V5, where DUX4-V5 signal appears as red fluorescence. Blue DAPI stain (4′,6-diamidino-2-phenylindole) shows HEK293 nuclei. The U7-asDUX4-7 and U7-asDUX4-8 constructs reduced DUX4-V5 protein staining compared to cells treated with non-targeting U7-snRNAs. Although the U7-asDUX4-4 sequence was functional in other assays, it did not reduce DUX4-V5 protein expression due to disruption of its binding site by the V5 tag as indicated in FIG. 3A. 40× objective. Scale bar, 50 microns. FIG. 3C shows the Myc-DUX4-fl construct used for western blot assay and possible mechanisms of DUX4 inhibition by lead U7-asDUX4 targeting of DUX4 (discussed in text). DUX4-s is a non-toxic potential isoform of DUX4 that lacks the C-terminal transactivation domain. FIG. 3D shows that Western blot results demonstrated reduced DUX4 protein in U7-asDUX4-treated cells compared to those transfected with non-targeting U7-snRNA. Two bands, 52 kDa and 60 kDa, appeared in western blots after using the anti-Myc antibody on CMV.myc-DUX4-transfected HEK293 cell extracts. The 60 kDa protein band was detected in untransfected cells and migrates at approximately the size of endogenous Myc protein. Consistent with prior immunofluorescence, cell death, and RNAscope results observed, U7-asDUX4 snRNAs reduced transfected DUX4 expression compared to non-targeting controls. Western blots were performed three times using protein extracts from three independent experiments (raw blots shown in FIG. 6). Tubulin was used as a normalizer. FIG. 3E shows quantification of the western blots of FIG. 3D. DUX4 protein signal intensity was significantly reduced in cells treated with U7-asDUX4-4 (87.4%±9.8) and U7-asDUX4-8 (84.7%±13.5), when compared to the nontargeting controls. The U7-asDUX4-5 and -7 target similar splice junction sites and U7-asDUX4-6 targets an intron1 SD site. These three constructs were tested to determine if any of them could induce DUX4-s production by altering correct splicing of full length DUX4 mRNA. No DUX4 short protein band (22 kDa) was detected using a western blot assay. ** P≤0.01, ANOVA; N=3 independent experiments, where each experiment was normalized to its respective non-targeting control, which was set a 100% DUX4 expression.

FIG. 4A-I shows U7-asDUX4 constructs reduce endogenous DUX4 and DUX4-associated biomarkers in FSHD patient-derived myotubes. FIG. 4A shows that FSHD 15A myotubes demonstrated higher amounts of DUX4 mRNA signal compared to cells treated with U7-asDUX4s. Arrows in panel FIG. 4A show an example of DUX4-positive indicate brown signal. DUX4 expression in FSHD 15A myotubes was reduced or absent in 15A cells transfected with U7-asDUX4-4 (FIG. 4B), U7-asDUX4-7 (FIG. 4C), and U7-asDUX4-8 (FIG. 4D). FIG. 4E shows very weak or absent signal was present in the unaffected 15V myotubes, which served as a negative control for RNAscope staining using DUX4 probe. FIG. 4F shows 15A myotubes stained with the housekeeping gene PPIB positive control for the RNAscope assay. FIG. 1G shows 15A myotubes stained with bacterial dapB gene probe, which served as a negative control for the assay. 100× objective. Scale bar, 20 microns. FIG. 1H shows quantification of DUX4 RNAscope signal, which was performed as described in Ref 30. 3-4 representative microscopic fields were counted from 3 independent experiments; each point represents quantification of one field. **P<0.01, ANOVA. DUX4 signal was absent or very low in unaffected 15V cells, as well as affected 15A cells transfected with lead U7-asDUX4 snRNA plasmids compared to untreated, affected 15A samples. **P≤0.01, ANOVA. FIG. 4I shows knockdown of DUX4-activated biomarkers by U7-asDUX4 sequences. Plots show significant reductions in ZSCAN4, PRAMEF12, MBD3L2, and TRIM43 in U7-asDUX4-treated FSHD 15A myotubes compared to controls transfected with non-targeting snRNA. N=4 independent experiments performed in triplicate or in some cases, duplicate. **P≤0.01, ANOVA.

FIG. 5A-C shows predicted splice site and splice enhancer/silencer sites on the DUX4 pre-mRNA using predictions from the Human Splice Finder 3.1 Tool. FIG. 5A shows U7-asDUX4 binding site locations overlapped with (FIG. 5B-C) predicted splice motifs.

FIG. 6A-C shows raw western blots 1 (FIG. 6A), 2 (FIG. 6B), and 3 (FIG. 6C).

FIG. 7 shows DUX4-s was not detected with nested RT-PCR. cDNA synthesis was primed with a previously described oligo-dT adaptor primer (Giesige et al., JCI Insight 2018;3(22):e123538). 3 μl of cDNA product was used as template in the first PCR reaction, and this product was then diluted 1/10 in the second round of PCR. The expected 569 bp band for DUX4-s was only found in the positive control (DUX4-s transfected cells) but absent in other samples. This experiment was repeated at least 4 times in DUX4-transfected HEK293s and in 15A human FSHD myotubes. DNA ladder, TrackIt 1 Kb Plus DNA Ladder. 1.5% Agarose gel.

| Sequence Name | SEQ ID NO: | Sequence |
|---|---|---|
| Oligo(dT)18 + adapter | 39 | GAATCGAGCACCAGTTACGCATGCCGAGGT |
| | | CGACTTCCTAGATTTTTTTTTTTTTTTTTTT |
| DUX4-s Forward primer | 40 | CAGAATGAGAGGTCACGCCAG |
| Adapter-Nested1 R | 41 | GAATCGAGCACCAGTTACGCATG |
| Adapter-Nested2 R | 42 | GAGCACCAGTTACGCATGCC |

FIG. 8 shows human15V FSHD myoblasts are efficiently transfected with a CMV.GFP plasmid. This experiment was performed to confirm that human myoblasts could be used to test the efficacy of DUX4 inhibition through U7-snRNA plasmid transfection.

FIG. 9 shows the development of RNAscope probes for in vivo use. RNAscope probes were designed to detect DUX4 and 5 DUX4-activated biomarkers, MBD3L2, PRAMEF12, LEUTX, ZSCAN4, and TRIM43. The top panel in FIG. 9 shows optimization of probes in vitro using DUX4 plasmid transfected HEK293s (brown stain). The bottom panel shows the colocalization of DUX4 and TRIM43 signal in serial sections from a FSHD patient muscle biopsy. Arrows show signal. The letters a, b, c help orient serial sections. Apparent signal from the DUX4 probe has been identified in 11 of 20 samples, and TRIM43 in 13 of 20 samples. Two samples had TRIM43 with no obvious DUX4 signal, while 7 samples showed no DUX4 or TRIM43 signal. These results demonstrate that DUX4 is not uniformly found in all myonuclei from FSHD patient biopsies. These results also demonstrate that RNAscope can be used for detecting DUX4 mRNA in vivo, in patients' samples that can be used as an outcome measure for clinical use.

### DETAILED DESCRIPTION

The disclosure provides a novel strategy to accomplish double homeobox protein 4 (DUX4) gene expression post-transcriptionally by repressing or inhibiting DUX4 protein production because the expression of DUX4 in muscle is known to cause muscular dystrophy including, but not limited to, facioscapulohumeral muscular dystrophy (FSHD). Thus, in some aspects, the products and methods described herein are used in treating, ameliorating, delaying the progression of, and/or preventing FSHD.

The DUX4 gene encodes an approximately 45kDA protein; see UniProtKB -Q9UBX2 (DUX4_HUMAN). De-repression of the DUX4 gene is involved in disease pathogenesis of FSHD. De-repression can occur through two known mechanisms: D4Z4 repeat contraction, or mutation in chromatin modifier genes SMCHD1 or DNMT3B. For the former, in unaffected subjects, the D4Z4 array consists of 11-100 repeats, while in FSHD1 patients, the array is reduced to 1-10 repeats (PubMed:19320656). Either condition can cause DNA hypomethylation at chromosome 4q35, thereby creating a chromosomal environment permissive for DUX4 expression.

DUX4 is located in D4Z4 macrosatellite repeats, which are epigenetically repressed in somatic tissues. D4Z4 chromatin relaxation in FSHD1 results in inefficient epigenetic repression of DUX4 and a variegated pattern of DUX4 protein expression in a subset of skeletal muscle nuclei. Ectopic expression of DUX4 in skeletal muscle activates the expression of stem cell and germline genes, and, when overexpressed in somatic cells, DUX4 can ultimately lead to cell death.

Each D4Z4 repeat unit has an open reading frame (named DUX4) that encodes two homeoboxes; the repeat-array and ORF is conserved in other mammals. The encoded protein has been reported to function as a transcriptional activator of numerous genes, including some considered to be FSHD disease biomarkers, including ZSCAN4, PRAMEF12, TRIM43, and MBD3L2 (PMID: 24861551). Contraction of the macrosatellite repeat causes autosomal dominant FSHD. Alternative splicing results in multiple transcript variants.

In some embodiments of the disclosure, the DUX4 nucleic acid and protein are provided. In some aspects, the nucleic acid encoding human DUX4 is set forth in the nucleotide sequence set forth in SEQ ID NO: 37. In some aspects, the amino acid sequence of human DUX4 is set forth in the amino acid sequence set forth in SEQ ID NO: 38. In various aspects, the methods of the disclosure also target isoforms and variants of the nucleotide sequence set forth in SEQ ID NO: 37. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 37 In some aspects, the methods of the disclosure target isoforms and variants of nucleic acids comprising nucleotide sequences encoding the amino acid sequence set forth in SEQ ID NO: 38. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to a nucleotide sequence that encodes the amino acid sequence set forth in SEQ ID NO: 38.

| SEQ ID NO: | Sequence |
|---|---|
| 37 | DUX4 NT: |
| | atggccctcccgacaccctcggacagcaccctccccgcggaagcccggggacgaggacggcgacgga |
| | gactcgtttggaccccgagccaaagcgaggccctgcgagcctgctttgagcggaacccgtacccgggcat |
| | cgccaccagagaacggctggcccaggccatcggcattccggagcccagggtccagatttggtttcagaat |
| | gagaggtcacgccagctgaggcagcaccggcgggaatctcggccctggcccgggagacgcggcccgc |
| | cagaaggccggcgaaagcggaccgccgtcaccggatcccagaccgccctgctcctccgagcctttgaga |
| | aggatcgctttccaggcatcgccgcccgggaggagctggccagagagacgggcctcccggagtccagg |
| | attcagatctggtttcagaatcgaagggccaggcacccgggacagggtggcagggcgcccgcgcaggc |
| | aggcggcctgtgcagcgcggcccccggcgggggtcaccctgctccctcgtgggtcgccttcgcccacacc |
| | ggcgcgtggggaacggggcttcccgcaccccacgtgccctgcgcgcctggggctctcccacagggggctt |
| | tcgtgagccaggcagcgagggccgcccccgcgctgcagcccagccaggccgcgccggcagaggggat |
| | ctcccaacctgccccggcgcgcggggatttcgcctacgccgccccggctcctccggacggggcgctctcc |
| | caccctcaggctcctcggtggcctccgcacccgggcaaaagccgggaggaccgggacccgcagcgcg |
| | acggcctgccgggcccctgcgcggtggcacagcctgggcccgctcaagcggggccgcagggccaagg |
| | ggtgcttgcgccacccacgtcccaggggagtccgtggtggggctggggccggggtccccaggtcgccgg |
| | ggcggcgtgggaaccccaagccggggcagctccacctccccagcccgcgcccccggacgcctccgcct |
| | ccgcgcggcaggggcagatgcaaggcatcccggcgccctcccaggcgctccaggagccggcgccctg |
| | gtctgcactcccctgcggcctgctgctggatgagctcctggcgagcccggagtttctgcagcaggcgcaacc |
| | tctcctagaaacggaggccccgggggagctggaggcctcggaagaggccgcctcgctggaagcacccc |
| | tcagcgaggaagaataccgggctctgctggaggagctttag |
| 38 | DUX4 AA: |
| | MALPTPSDSTLPAEARGRGRRRRLVWTPSQSEALRACFERNPYPGIATRER |
| | LAQAIGIPEPRVQIWFQNERSRQLRQHRRESRPWPGRRGPPEGRRKRTAV |
| | TGSQTALLLRAFEKDRFPGIAAREELARETGLPESRIQIWFQNRRARHPGQG |
| | GRAPAQAGGLCSAAPGGGHPAPSWVAFAHTGAWGTGLPAPHVPCAPGAL |
| | PQGAFVSQAARAAPALQPSQAAPAEGISQPAPARGDFAYAAPAPPDGALSH |
| | PQAPRWPPHPGKSREDRDPQRDGLPGPCAVAQPGPAQAGPQGQGVLAPP |
| | TSQGSPWWGWGRGPQVAGAAWEPQAGAAPPPQPAPPDASASARQGQM |
| | QGIPAPSQALQEPAPWSALPCGLLLDELLASPEFLQQAQPLLETEAPGELEA |
| | SEEAASLEAPLSEEEYRALLEEL |

There is currently no treatment for FSHD, and despite its relative abundance among the muscular dystrophies, very few FSHD-targeted translational studies have been published. Several FSHD candidate genes have been identified, but numerous recent studies support that the primary contributor to FSHD pathogenesis is the pro-apoptotic DUX4 gene, which encodes a transcription factor. Thus, in the simplest terms, DUX4-overexpression is a primary pathogenic insult underlying FSHD (Chen et al., (2016) Mol Ther 24, 1405-1411; Ansseau et al. (2017) Genes (Basel) 8; Lek et al. (2020) Sci Trans! Med 12; Himeda et al. (2016) Mol Ther 24, 527-535; DeSimone et al. (2019) Sci Adv 5, 12; Lim et al. (2020) Proc Natl Acad Sci U S A 117, 16509-16515; Wallace et al. (2018), supra; Rojas et al. (2020) J Pharmacol Exp Ther. Sep; 374(3):489-498).

In some embodiments, the disclosure provides antisense RNA (asRNA), also referred to as antisense transcript, natural antisense transcript, or antisense oligonucleotide. Antisense RNA is a single stranded RNA that is complementary to a protein coding messenger RNA (mRNA) with which it hybridizes, and thereby blocks its translation into protein. The primary function of asRNA is regulating gene expression. Antisense RNAs may also be produced synthetically, as described herein, are used in downregulating DUX4 expression.

The disclosure provides novel constructs and methods to accomplish DUX4 knockdown or silencing by using U7-antisense(as)DUX4 snRNAs. U7-as snRNAs are transcribed in the nucleus, then exported to the cytoplasm, where they assemble with Sm and Lsm proteins. The assembled U7-snRNP can remain in the cytoplasm or be imported back into the nucleus. In the nucleus, they are associated with splicing machinery, while in the cytoplasm they associate with P bodies, which normally function in mRNA turnover (Liu et al. (2007). PNAS 104(28), 11655-11659). Similarly, mRNAs can be detected in both the nucleus and the cytoplasm, as they are transcribed and matured in the nucleus, and then transported to the cytoplasm for translation.

In some embodiments, the disclosure provides a gene therapy approach to treat FSHD by downregulating or inhibiting expression of the toxic DUX4 gene in muscle. The full-length DUX4 gene product causes cell death and muscle toxicity and, thus, the FSHD therapy described herein is designed to inhibit full length DUX4 expression. In some aspects described herein, DUX4 inhibition is accomplished using U7-snRNA antisense expression cassettes (called U7-asDUX4). These non-coding RNAs were designed to inhibit production or maturation of the full length DUX4 pre-mRNA by masking the DUX4 start codon, splice sites, or polyadenylation signal. The U7-asDUX4 constructs have three major features: a stabilizing hairpin structure at one end, a binding site for Sm proteins, and an antisense region that can be modified to target any gene of interest, e.g., DUX4.

Some constructs identified herein target the exon 1/intron1 junction (i.e., U7-asDUX4-4 and -7), or the DUX4 poly A signal (PAS) (i.e., U7-asDUX4-8). Targeting the splice junction is a new approach although the use of antisense sequences to bind the DUX4 PAS was previously demonstrated using chemically synthesized ASOs, which were shown to reduce DUX4 and DUX4-activated biomarkers in vitro and in vivo (Vanderplanck et al. (2011) PLoS One 6, e26820; Marsollier et al. (2016) Hum Mol Genet 25, 1468-1478; Chen, et al. (2016) Mol Ther 24, 1405-1411; Ansseau et al. (2017) Genes (Basel) 8). The U7-asDUX4 sequences described herein are unique, novel, and distinct from ASOs because they incorporate additional sequences to recruit Sm and Lsm proteins, and are expressed in vivo from a promoter. Polyadenylation is an important process required for stabilizing nascent mRNAs and coordinating mRNA transit through nuclear pores to the cytoplasm for translation. Chemically synthesized DNA-based ASOs may operate by forming DNA:RNA hybrids and activating RNAse H against the target transcript, but it is also possible that published ASO sequences designed to base pair with the DUX4 PAS could operate by masking the signal and preventing polyadenylation, thereby leading to DUX4 mRNA destabilization.

In some embodiments, the disclosure provides nucleic acids comprising nucleotide sequences encoding U7 snRNAs (U7-asDUX4) targeting DUX4 and inhibiting the expression of DUX4. The disclosure includes various nucleic acids comprising, consisting essentially of, or consisting of the various nucleotide sequences described herein. In some aspects, the nucleic acid comprises the nucleotide sequence. In some aspects, the nucleic acid consists essentially of the nucleotide sequence. In some aspects, the nucleic acid consists of the nucleotide sequence.

Thus, in some aspects, the disclosure includes a nucleic acid comprising a polynucleotide encoding an inhibitory RNA to prevent and inhibit the expression of the DUX4 gene. The inhibitory RNA comprises an antisense sequence, which inhibits the expression of DUX4. The sequences set forth in SEQ ID NOs: 1-18 are DNA sequences encoding the U7 snRNAs which prevent and inhibit the expression of the DUX4 gene.

In some aspects, the term “U7-asDUX4” is used interchangeably herein to mean a nucleotide sequence set forth in any one of SEQ ID NOs: 1-18. In some other aspects, the disclosure includes a nucleic acid comprising a polynucleotide encoding a DUX4 antisense, e.g. U7-asDUX4, targeting a DUX4 sequence comprising the nucleotide sequence set forth in any one of SEQ ID NOs: 19-36.

In some aspects, the term “U7-asDUX4” is used interchangeably herein to mean a nucleotide sequence encoding a U7 double homeobox 4 (DUX4) antisense sequence that specifically hybridizes to a DUX4 target nucleotide sequence set forth in any one of SEQ ID NOs: 19-36.

In some aspects, therefore, the disclosure includes (1) a nucleic acid comprising any one of the nucleotide sequences set forth in SEQ ID NOs: 1-18, or a variant thereof comprising at least about 90%, 91%, 92%, 93%, 94%, 95%, 96%, 97%, 98%, or 99% identity to any one of the sequences set forth in any of SEQ ID NOs: 1-18; and (2) a nucleic acid comprising a nucleotide sequence that encodes an snRNA that targets any one of the nucleotide sequences set forth in SEQ ID NOs: 19-36.

In some aspects, the disclosure includes a nucleic acid comprising a nucleotide sequence comprising any one of the sequences set forth in SEQ ID NOs: 1-18, or a variant thereof comprising at least about 90%, 91%, 92%, 93%, 94%, 95%, 96%, 97%, 98%, or 99% identity to any one of the sequences set forth in SEQ ID NOs: 1-18 under the control of a U7 promoter. In some aspects, the disclosure includes a nucleic acid comprising any one of the nucleotide sequences set forth in SEQ ID NOs: 1-18 under the control of another promoter including, but not limited to a muscle-specific promoter.

In some aspects, therefore, the disclosure includes a nucleotide sequence that encodes an snRNA that binds to any one of the target sequences set forth in SEQ ID NOs: 19-36 under the control of a U7 promoter. In some aspects, the disclosure includes a nucleic acid comprising a nucleotide sequence that binds to any one of the target sequences set forth in SEQ ID NOs: 19-36 under the control of another promoter including, but not limited to a muscle-specific promoter.

Exemplary nucleotide sequences used in snRNA targeting of DUX4 described herein include, but are not limited to, those identified in Table 1 below. Various properties of these nucleotide sequences are set out in Table 2 below.

TABLE 1
Nucleotide sequences-U7-asDUX4 antisense sequences and DUX4 target sequences

| | | SEQ | | SEQ |
|---|---|---|---|---|
| U7-as | DNA sequences which encode U7- | ID | | ID |
| DUX4 | asDUX4 antisense sequences | NOS: | DUX4 target sequences | NOS: |
| 1 | GCTGAGGGGTGCTTCCAGCGAGGCGGCCTCTT | 1 | GGAAGAGGCCGCCTCGCTGGAAGCACCCCTCAGC | 19 |
| | CC | | | |
| 2 | AGGCCTCCAGCTCCCCCGGGGCCTCCGTTTCT | 2 | TAGAAACGGAGGCCCCGGGGGAGCTGGAGGCCT | 20 |
| | A | | | |
| 3 | TGCAGAAACTCCGGGCTCGCCAGGAGCTCATC | 3 | GATGAGCTCCTGGCGAGCCCGGAGTTTCTGCA | 21 |
| 4 | AACCCCGCGTCCTAAAGCTCCTCCAGCAGAGC | 4 | GAAGAATACCGGGCTCTGCTGGAGGAGCTTTAGGACGC | 22 |
| | CCGGTATTCTTC | | GGGGTT | |
| 5 | GCTCCTCCAGCAGAGCCCGGTATTCTTCCTCGC | 5 | TGGAAGCACCCCTCAGCGAGGAAGAATACCGGGCTCTG | 23 |
| | TGAGGGGTGCTTCCA | | CTGGAGGAGC | |
| 6 | CGAACCACCCGACCCCGTCCCAACCCCGCGTC | 6 | TAGGACGCGGGGTTGGGACGGGGTCGGGTGGTTCG | 24 |
| | CTA | | | |
| 7 | AGCTCCTCCAGCAGAGCCCGGTATTCTTCCTC | 7 | GAGGAAGAATACCGGGCTCTGCTGGAGGAGCT | 25 |
| 8 | GGAGGGGGCATTTTAATATATCTCTGAACT | 8 | AGTTCAGAGATATATTAAAATGCCCCCTCC | 26 |
| 9 | GTGCTGTCCGAGGGTGTCGGGAGGGCCAT | 9 | ATGGCCCTCCCGACACCCTCGGACAGCAC | 27 |
| 10 | GGCTTCCGCGGGGAGGGTGCTGTCCGAGGGT | 10 | ATGGCCCTCCCGACACCCTCGGACAGCACCCTCCCCGC | 28 |
| | GTCGGGAGGGCCAT | | GGAAGCC | |
| 11 | GAACCACCCGACCCCGTCCCAACCCCGCGTCC | 11 | GCTGGAGGAGCTTTAGGACGCGGGGTTGGGACGGGGT | 29 |
| | TAAAGCTCCTCCAGC | | CGGGTGGTTC | |
| 12 | GAACCACCCGACCCCGTCCCAACCCCGCGTCC | 12 | GAAGAATACCGGGCTCTGCTGGAGGAGCTTTAGGACGC | 30 |
| | TAAAGCTCCTCCAGCAGAGCCCGGTATTCTTC | | GGGGTTGGGACGGGGTCGGGTGGTTC | |
| 13 | GTGCGCAGTAGGCGGCCCACCTGCTGGTACCT | 13 | AGGTACCAGCAGGTGGGCCGCCTACTGCGCAC | 31 |
| 14 | CGCGCAGGTCTAGTCAGGAAGCGGGCAAAGAC | 14 | TCTGTCTTTGCCCGCTTCCTGACTAGACCTGCGCG | 32 |
| | AGA | | | |
| 15 | CGGGGTGCGCACTGCGCGCAGGTCTAGTCAGG | 15 | CAAAAGCATACCTCTGTCTGTCTTTGCCCGCTTCCTGAC | 33 |
| | AAGCGGGCAAAGACAGACAGAGGTATGCTTTTG | | TAGACCTGCGCGCAGTGCGCACCCCG | |
| 16 | GCACGTCAGCCGGGGTGCGCACTGCGCGCAG | 16 | CCTGACTAGACCTGCGCGCAGTGCGCACCCCGGCTGAC | 34 |
| | GTCTAGTCAGG | | GTGC | |
| 17 | GGGGGCATTTTAATATATCTCTGAACTAATCATC | 17 | CTCCTGGATGATTAGTTCAGAGATATATTAAAATGCCCC | 35 |
| | CAGGAG | | C | |
| 18 | GGAGGGGGCATTTTAATATATCTCTGAACTAAT | 18 | CCTGGATTAGAGTTACATCTCCTGGATGATTAGTTCAGA | 36 |
| | CATCCAGGAGATGTAACTCTAATCCAGG | | GATATATTAAAATGCCCCCTCC | |

TABLE 2
Various properties of the nucleic acids of the disclosure

| U7-as | | | HSF score | HSF score | | | |
|---|---|---|---|---|---|---|---|
| DUX4 | Length | GC % | for SE motif | for SD/AS | Target site | #Nucleotide | Function |
| 1 | 34 | 70.6 | 82.38 | 82.75 | Ex1 SA | 1206-1239 | Interfering with the correct splicing |
| 2 | 33 | 69.7 | 85.74 | 72.93 | Ex1 SA | 1172-1204 | Interfering with the correct splicing |
| 3 | 32 | 62.5 | 87.83 | — | SE | 1123-1154 | Targeting ex1 SE |
| 4 | 44 | 59.1 | 83.34 | 78.88-78.09 | Ex1 SA-Int1 | 1243-1286 | Interfering with the correct splicing |
| | | | | | SD | | |
| 5 | 48 | 62.5 | 86.47 | 82.75 | Ex1 SA | 1223-1270 | Interfering with the correct splicing |
| 6 | 35 | 71.4 | 82.48 | 78.09 | Int1 SD | 1273-1307 | Interfering with the correct splicing |
| 7 | 32 | 59.4 | 83.34 | 78.88 | Ex1 SA | 1240-1271 | Interfering with the correct splicing |
| 8 | 30 | 40 | 77.77 | — | PolyA | 2027-2056 | polyA destabilization |
| 9 | 29 | 69 | 91.31 | — | Start codon | 1-29 | Translation blocking |
| 10 | 45 | 73.3 | 91.31 | — | Start codon | 1-45 | Translation blocking |
| 11 | 47 | 68.1 | 82.48 | 78.09 | Int1 SD | 1260-1306 | Interfering with the correct splicing |
| 12 | 64 | 64.1 | 83.34 | 78.88-78.09 | Ex1 SA-Int1 | 1243-1306 | Interfering with the correct splicing |
| | | | | | SD | | |
| 13 | 32 | 68.8 | 89.4 | 83.13-88.33 | Ex2 SA- Int2 | 1500-1531 | Interfering with the correct splicing |
| | | | | | SD | | |
| 14 | 35 | 60 | 90.18 | 81.66 | Ex3 SA | 1847-1881 | Interfering with the correct splicing |
| 15 | 65 | 60 | 90.18 | 81.66 | Ex3 SA | 1831-1895 | Interfering with the correct splicing |
| 16 | 71.4 | 76.1 | 90.18 | 81.66 | Ex3 SA | 1864-1905 | Interfering with the correct splicing |
| 17 | 40 | 61.4 | 83.84 | — | PolyA | 2014-2053 | polyA destabilization |
| 18 | 41 | 66.1 | 88.26 | — | PolyA | 1996-2056 | polyA destabilization |

The length in column 2 refers to the number of the nucleotides that encode each snRNA. The Human Splicing Finder (HSF) scores are automatically made by the HSF program's algorithm for predicting splicing sites on DUX4 mRNA, have been used to design snRNAs in this study. The HSF score has a range of 0-100, of which a higher score indicates a stronger splice prediction. The nucleotide column refers to the position on the DUX4 cDNA nucleotide sequence used as a reference sequence for designing the snRNAs.

The DNA nucleotide sequences set out above in Tables 1 and 2 (1) encode the RNA antisense sequences for targeting DUX4, or (2) are the target sequence site for the DUX4 snRNA.

In some aspects, the disclosure provides snRNAs or U-RNAs which inhibit or interfere with the expression of the DUX4 gene. In some aspects, the snRNAs are driven by or under the control of a human or a murine U7 promoter, i.e., U7snRNAs. In some aspects, the snRNAs are under the control of any other promoter including, but not limited to, for example, a tissue-specific or a muscle-specific promoter.

In some embodiments, the products and methods of the disclosure comprise nucleic acids encoding small nuclear ribonucleic acids (snRNAs), also commonly referred to as U-RNAs, to downregulate or inhibit DUX4 expression. snRNAs are a class of small RNA molecules that are found within the splicing speckles and Cajal bodies of the cell nucleus in eukaryotic cells. Small nuclear RNAs are associated with a set of specific proteins, and the complexes are referred to as small nuclear ribonucleoproteins (snRNP, often pronounced “snurps”). Each snRNP particle is composed of a snRNA component and several snRNP-specific proteins (including Sm proteins, a family of nuclear proteins). The snRNAs, along with their associated proteins, form ribonucleoprotein complexes (snRNPs), which bind to specific sequences on the pre-mRNA substrate. They are transcribed by either RNA polymerase II or RNA polymerase III. snRNAs are often divided into two classes based upon both common sequence features and associated protein factors, such as the RNA-binding LSm proteins. The first class, known as Sm-class snRNA, consists of U1, U2, U4, U4atac, U5, U7, U11,and U12. Sm-class snRNA are transcribed by RNA polymerase II. The second class, known as Lsm-class snRNA, consists of U6 and U6atac. Lsm-class snRNAs are transcribed by RNA polymerase III and never leave the nucleus, in contrast to Sm-class snRNA. In some aspects, the disclosure includes the production and administration of an AAV vector comprising U7 snRNA for the delivery of DUX4 antisense sequences.

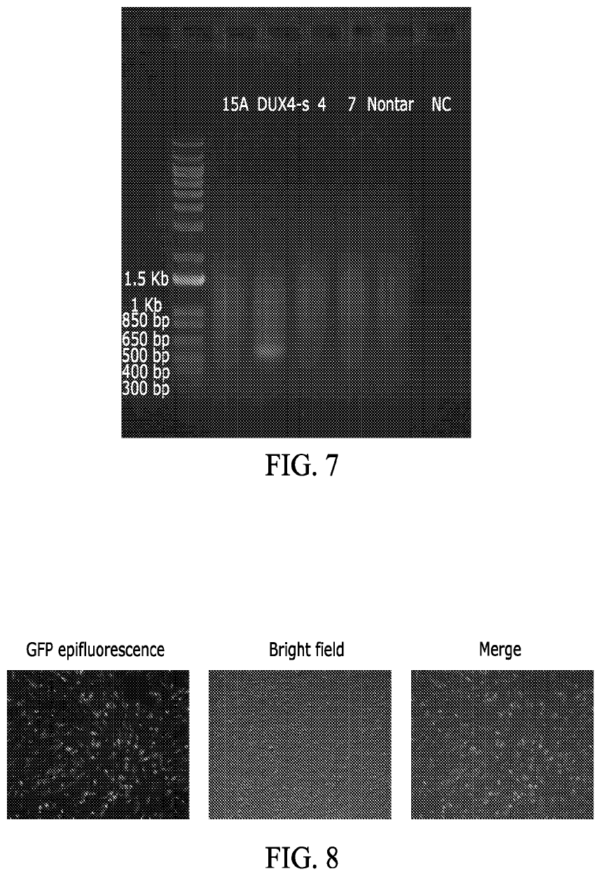

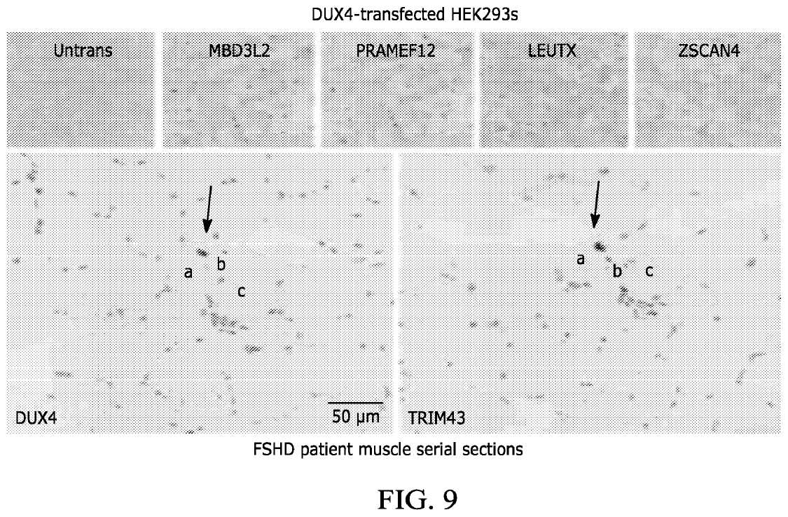

In some aspects, the disclosure uses U7 snRNA molecules to inhibit, knockdown, or interfere with gene expression. U7 snRNA is normally involved in histone pre-mRNA 3′ end processing but, in some aspects, is converted into a versatile tool for splicing modulation or as antisense RNA that is continuously expressed in cells (Goyenvalle et al., Science 306(5702): 1796-9 (2004)). By replacing the wild-type U7 Sm binding site with a consensus sequence derived from spliceosomal snRNAs, the resulting RNA assembles with the seven Sm proteins found in spliceosomal snRNAs (FIG. 7). As a result, this U7 Sm OPT RNA accumulates more efficiently in the nucleoplasm and will no longer mediate histone pre-mRNA cleavage, although it can still bind to histone pre-mRNA and act as a competitive inhibitor for wild-type U7 snRNPs. By further replacing the sequence binding to the histone downstream element with one complementary to a particular target in a splicing substrate, it is possible to create U7 snRNAs capable of modulating specific splicing events. The advantage of using U7 derivatives is that the antisense sequence is embedded into a small nuclear ribonucleoprotein (snRNP) complex. Moreover, when embedded into a gene therapy vector, these small RNAs can be permanently expressed inside the target cell after a single injection [Gorman et al., Proc Natl Acad Sci. 28; 95(9): 4929-34 (1998); Goyenvalle et al., Science. 3;306(5702):1796-9 (2004); Levy et al., Eur. J. Hum. Genet. 18(9): 969-70 (2010); Wein et al., Hum. Mutat. 31(2): 136-42, (2010); Wein et al., Nat. Med. 20(9): 992-1000 (2014)]. Use of U7 for altering the expression of the CUG repeat has been tested in vitro in a DM1 patient cell line [Francois et al., Nat. Struct. Mol. Biol. 18(1): 85-7 (2011)] where it has been shown that a U7 RNA targeting the CUG repeat results in decreased amounts of DUX4 related foci and correction of the aberrant splicing pattern; however, this approach was based on lentivirus and was never pursued further in vivo. The potential of U7snRNA systems in neuromuscular disorders using an AAV approach has been investigated in vivo (AAV.U7) [Gorman et al., Proc Natl Acad Sci. 28;95(9):4929-34 (1998); Goyenvalle et al., Science. 3; 306(5702):1796-9 (2004); Levy et al., Eur. J. Hum. Genet. 18(9): 969-70 (2010); Wein et al., Hum. Mutat. 31(2): 136-42 (2010); Wein et al., Nat. Med. 20(9): 992-1000 (2014)].

U7 snRNA is normally involved in histone pre-mRNA 3′ end processing, but also is used as a versatile tool for splicing modulation or as antisense RNA that is continuously expressed in cells. One advantage of using U7 derivatives is that the antisense sequence is embedded into a small nuclear ribonucleoprotein (snRNP) complex. Moreover, when embedded into a gene therapy vector, these small RNAs can be permanently expressed inside the target cell after a single injection.

In some aspects, the disclosure includes a nanoparticle, extracellular vesicle, exosome, or vector comprising any of the nucleic acids of the disclosure or a combination of any one or more thereof. In some aspects, one or more nucleic acids are combined into a single nanoparticle, extracellular vesicle, exosome, or vector. In some aspects, the nanoparticle is a liposome or micelle.

In some embodiments, the disclosure includes a vector comprising any of the nucleic acids or a combination of any of the nucleic acids described herein. Embodiments of the disclosure utilize vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein.

In some embodiments, the disclosure includes vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein or combinations of the nucleic acids.

In some embodiments, the vectors are AAV vectors. In some aspects, the vectors are single stranded AAV vectors. In some aspects the AAV is recombinant AAV (rAAV). In some aspects, the rAAV lack rep and cap genes. In some aspects, rAAV are self-complementary (sc)AAV.

Thus, in some aspects, the viral vector is an adeno-associated virus (AAV), such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), AAVrh.10 (i.e., an AAV containing AAVrh.10 ITRs and AAVrh.10 capsid proteins), AAV11 (i.e., an AAV containing AAV11 ITRs and AAV11 capsid proteins), AAV12 (i.e., an AAV containing AAV12 ITRs and AAV12 capsid proteins), AAV13 (i.e., an AAV containing AAV13 ITRs and AAV13 capsid proteins), AAV-anc80, AAV rh.74, AAV rh.8, AAVrh.10, or AAV-B1.

Some embodiments of the disclosure, therefore, include an rAAV genome comprising a nucleic acid comprising the nucleotide sequence set out in any of SEQ ID NOs: 1-18 or a variant thereof comprising a nucleotide sequence having at least about 90% sequence identity to the sequence set out in any of SEQ ID NOs: 1-18 as disclosed herein in the detailed description. Additionally, some embodiments of the disclosure include an rAAV genome comprising a nucleic acid comprising a nucleotide sequence which binds to the target sequence set out in any of SEQ ID NOs: 19-36 as disclosed herein in the detailed description.

AAV is a replication-deficient parvovirus, the single-stranded DNA genome of which is about 4.7 kb in length including 145 nucleotides in inverted terminal repeat (ITRs). There are multiple serotypes of AAV. The nucleotide sequences of the genomes of the AAV serotypes are known. For example, the complete genome of AAV1 is provided in GenBank Accession No. NC_002077; the complete genome of AAV2 is provided in GenBank Accession No. NC_001401 and Srivastava et al., J. Virol., 45: 555-564 {1983); the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV6 is provided in GenBank Accession No. NC_00 1862; at least portions of AAV7 and AAV8 genomes are provided in GenBank Accession Nos. AX753246 and AX753249, respectively (see also U.S. Pat. Nos. 7,282,199 and 7,790,449 relating to AAV8); the AAV9 genome is provided in Gao et al., J. Virol., 78: 6381-6388 (2004); the AAV10 genome is provided in Mol. Ther., 13(1): 67-76 (2006); the AAV11 genome is provided in Virology, 330(2): 375-383 (2004); the AAV12 genome is provided in J Virol. 2008 Feb; 82(3):1399-406; and the AAV13 genome is provided in J Virol 2008; 82:8911. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.7 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may be lyophilized and AAV-infected cells are not resistant to superinfection.

In some embodiments, DNA plasmids of the disclosure are provided which comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, AAVrh.10, and AAV-B1. In some aspects, AAV DNA in the rAAV genomes is from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, AAVrh.10, and AAV-B1. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some embodiments, recombinant AAV genomes of the disclosure comprise one or more AAV ITRs flanking a polynucleotide sequence, for example, one or more an antisense sequences that bind to key exon definition elements in the pre-mRNA. Thus, in some embodiments, rAAV genomes of the disclosure comprise one or more AAV ITRs flanking a polynucleotide encoding, for example, one or more DUX4 antisense sequences. Commercial providers such as Ambion Inc. (Austin, TX), Darmacon Inc. (Lafayette, CO), InvivoGen (San Diego, CA), and Molecular Research Laboratories, LLC (Herndon, VA) generate custom inhibitory RNA molecules. In addition, commercial kits are available to produce custom siRNA molecules, such as SILENCERTM siRNA Construction Kit (Ambion Inc., Austin, TX) or psiRNA System (InvivoGen, San Diego, CA).

Thus, in some embodiments, a recombinant AAV genome of the disclosure comprises one or more AAV ITRs flanking at least one DUX4-targeted polynucleotide construct. In some embodiments, the polynucleotide is an snRNA, a polynucleotide encoding the snRNA, or a polynucleotide encoding an snRNA designed to bind to the target sequence. In some aspects, the polynucleotide encoding the snRNA is administered with other polynucleotide constructs targeting DUX4.

In various aspects, promoters are used to permit tissue specific expression. In some aspects, the snRNA is expressed under various promoters including, but not limited to, such promoters as a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, AAVrh.10, and AAV-B1. As set out herein above, the nucleotide sequences of the genomes of various AAV serotypes are known in the art.

In some embodiments, the viral vector is a pseudotyped AAV, containing ITRs from one AAV serotype and capsid proteins from a different AAV serotype. In some embodiments, the pseudo-typed AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is AAV2/1 (i.e., an AAV containing AAV2 ITRs and AAV1 capsid proteins).

In some embodiments, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAV-anc80, AAVrh74, AAVrh.8, or AAVrh.10, AAV10, AAV11, AAV12, AAV13, or AAV-B1. Other types of rAAV variants, for example rAAV with capsid mutations, are also contemplated. See, for example, Marsic et al., Molecular Therapy, 22(11): 1900-1909 (2014). As set out herein above, the nucleotide sequences of the genomes of various AAV serotypes are known in the art.

In some embodiments, packaging cells are provided. Packaging cells are created in order to have a cell line that stably expresses all the necessary components for AAV particle production. Retroviral vectors are created by removal of the retroviral gag, pol, and env genes. These are replaced by the therapeutic gene. In order to produce vector particles, a packaging cell is essential. Packaging cell lines provide all the viral proteins required for capsid production and the virion maturation of the vector. Thus, packaging cell lines are made so that they contain the gag, pot and env genes. Following insertion of the desired gene into in the retroviral DNA vector, and maintenance of the proper packaging cell line, it is now a simple matter to prepare retroviral vectors

For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

In some embodiments, the disclosure includes a composition comprising any of the nucleic acids or any of the vectors described herein in combination with a diluent, excipient, or buffer.

In some embodiments, therefore, a method of generating a packaging cell to create a cell line that stably expresses all the necessary components for AAV particle production is provided. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy et al., 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbiol. and Immunol. 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol . Cell. Biol. 5:3251 (1985); McLaughlin et al., J. Virol., 62:1963 (1988); and Lebkowski et al., 1988 Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/U598/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Human Gene Therapy, 4:609-615 (1993); Clark et al., Gene Therapy, 3:1124-1132 (1996); U.S. Pat. Nos. 5,786,211; 5,871,982; 6,258,595; and McCarty, Mol. Ther., 16(10): 1648-1656 (2008). The foregoing documents are hereby incorporated by reference in their entirety herein, with particular emphasis on those sections of the documents relating to rAAV production. The production and use of various types of rAAV are specifically contemplated and exemplified. Recombinant AAV (i.e., infectious encapsidated rAAV particles) are thus provided herein. In some aspects, genomes of the rAAV lack AAV rep and cap genes; that is, there is no AAV rep or cap DNA between the ITRs of the genomes of the rAAV. In some embodiments, the AAV is a recombinant linear AAV (rAAV), a single-stranded AAV (ssAAV), or a recombinant self-complementary AAV (scAAV).

The disclosure thus provides in some embodiments packaging cells that produce infectious rAAV. In one embodiment, packaging cells are stably transformed cancer cells, such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV, in some aspects, are purified by methods standard in the art, such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In some embodiments, the disclosure provides a composition or compositions comprising a nucleic acid or a vector, e.g., such as a viral vector, as described herein. Thus, compositions comprising delivery vehicles (such as rAAV) described herein are provided. In various aspects, such compositions also comprise a pharmaceutically acceptable carrier. In various aspects, such compositions also comprise other ingredients, such as a diluent, excipients, and/or adjuvant. Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions, such as sodium; and/or nonionic surfactants, such as Tween, pluronics or polyethylene glycol (PEG).

In some aspects, the nucleic acids are introduced into a vector for delivery. In some aspects, the vector for delivery is an AAV or an rAAV. Thus, embodiments of the disclosure include an rAAV genome comprising a nucleic acid comprising (i) a nucleotide sequence set out in any of SEQ ID NOs: 1-18, or a variant thereof comprising at least about 90% sequence identity to the sequence set out in any of SEQ ID NOs: 1-18; or (ii) a nucleic acid comprising a nucleotide sequence which encodes a DUX4 asRNA which binds to a DUX4 target sequence set out in any of SEQ ID NOs: 19-36.

In some other aspects, the nucleic acids are introduced into the cell via non-vectorized delivery. Thus, in an embodiment, the disclosure includes non-vectorized delivery of a nucleic acid encoding the DUX4 asRNAs. In some aspects, in this context, synthetic carriers able to form complexes with nucleic acids, and protect them from extra- and intracellular nucleases, are an alternative to viral vectors. The disclosure includes such non-vectorized delivery. The disclosure also includes compositions comprising any of the constructs described herein alone or in combination.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg) (e.g., 1×10⁷ vg, 1×10⁸ vg, 1×10⁹ vg, 1×10¹⁰ vg, 1×10¹¹ vg, 1×10¹² vg, 1×10¹³ vg, and 1×10¹⁴ vg, respectively).

In some aspects, therefore, the disclosure provides a method of delivering to a cell or to a subject any one or more nucleic acids comprising (i) a polynucleotide encoding a U7-asDUX4 antisense comprising the nucleotide sequence set forth in any one of SEQ ID NOs: 1-18, or a variant thereof comprising at least about 90%, 91%, 92%, 93%, 94%, 95%, 96%, 97%, 98%, or 99% identity to any one of the sequences set forth in SEQ ID NOs: 1-18, and/or (ii) a polynucleotide encoding a U7-asDUX4 antisense targeting a DUX4 sequence comprising the nucleotide sequence set forth in any one of SEQ ID NOs: 19-36.

In some aspects, the method comprises administering to a cell or to a subject an AAV comprising any one or more nucleic acids comprising (i) a polynucleotide encoding a U7-asDUX4 antisense construct comprising the nucleotide sequence set forth in any one of SEQ ID NOs: 1-18, or a variant thereof comprising at least about 90%, 91%, 92%, 93%, 94%, 95%, 96%, 97%, 98%, or 99% identity to any one of the sequences set forth in SEQ ID NOs: 1-18, and/or (ii) a polynucleotide encoding a U7-asDUX4 antisense targeting a DUX4 sequence comprising the nucleotide sequence set forth in any one of SEQ ID NOs: 19-36.

In yet another aspect, the disclosure provides a method of decreasing expression of the DUX4 gene or decreasing the expression of functional DUX4 in a cell or a subject, wherein the method comprises contacting the cell or the subject with any one or more nucleic acids comprising (i) a polynucleotide encoding a U7-asDUX4 antisense comprising the nucleotide sequence set forth in any one of SEQ ID NOs: 1-18, or a variant thereof comprising at least about 90%, 91%, 92%, 93%, 94%, 95%, 96%, 97%, 98%, or 99% identity to any one of the sequences set forth in SEQ ID NOs: 1-18, and/or (ii) a polynucleotide encoding a U7-asDUX4 antisense targeting a DUX4 sequence comprising the nucleotide sequence set forth in any one of SEQ ID NOs: 19-36.

In some aspects, the method comprises delivering the nucleic acids in one or more AAV vectors. In some aspects, the method comprises delivering the nucleic acids to the cell in non-vectorized delivery.

In some aspects, expression of DUX4 or the expression of functional DUX4 is decreased in a cell or in a subject by the methods provided herein by at least or about 5, about 10, about 15, about 20, about 25, about 30, about 35, about 40, about 45, about 50, about 55, about 60, about 65, about 70, about 75, about 80, about 85, about 90, about 95, about 96, about 97, about 98, about 99, or 100 percent.

In some aspects, the disclosure provides AAV transducing cells for the delivery of nucleic acids encoding the U7-asDUX4 antisense constructs as described herein. Methods of transducing a target cell with rAAV, in vivo or in vitro, are included in the disclosure. The methods comprise the step of administering an effective dose, or effective multiple doses, of a composition comprising a rAAV of the disclosure to a subject, including an animal (such as a human being) in need thereof. If the dose is administered prior to development of the muscular dystrophy, the administration is prophylactic. If the dose is administered after the development of the muscular dystrophy, the administration is therapeutic. In embodiments of the disclosure, an effective dose is a dose that alleviates (eliminates or reduces) at least one symptom associated with the muscular dystrophy being treated, that slows or prevents progression of the muscular dystrophy, that slows or prevents progression of the muscular dystrophy, that diminishes the extent of disease, that results in remission (partial or total) of the muscular dystrophy, and/or that prolongs survival. In some aspects, the muscular dystrophy is FSHD.

Combination therapies are also contemplated by the disclosure. Combination as used herein includes simultaneous treatment or sequential treatments. Combinations of methods of the disclosure with standard medical treatments (e.g., corticosteroids and/or immunosuppressive drugs) or with other inhibitory RNA constructs are specifically contemplated, as are combinations with other therapies such as those disclosed in International Publication No. WO 2013/016352, which is incorporated by reference herein in its entirety.

Administration of an effective dose of the compositions may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. Route(s) of administration and serotype(s) of AAV components of rAAV (in particular, the AAV ITRs and capsid protein) of the disclosure may be chosen and/or matched by those skilled in the art taking into account the disease state being treated and the target cells/tissue(s), such as cells that express DUX4. In some embodiments, the route of administration is intramuscular. In some embodiments, the route of administration is intravenous.

In some aspects, actual administration of rAAV of the present disclosure may be accomplished by using any physical method that will transport the rAAV recombinant vector into the target tissue of an animal. Administration according to the disclosure includes, but is not limited to, injection into muscle, the bloodstream, the central nervous system, and/or directly into the brain or other organ. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. In some aspects, proper fluidity is maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

The term “transduction” is used to refer to the administration/delivery of one or more of the nucleic acids described herein to a recipient cell either in vivo or in vitro, via a replication-deficient rAAV of the disclosure resulting in expression of the DUX4 miRNA by the recipient cell.

In one aspect, transduction with rAAV is carried out in vitro. In one embodiment, desired target cells are removed from the subject, transduced with rAAV and reintroduced into the subject. Alternatively, syngeneic or xenogeneic cells can be used where those cells will not generate an inappropriate immune response in the subject.

Suitable methods for the transduction and reintroduction of transduced cells into a subject are known in the art. In one embodiment, cells are transduced in vitro by combining rAAV with cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

The disclosure provides methods of administering an effective dose (or doses, administered essentially simultaneously or doses given at intervals) of rAAV that comprise DNA that encodes microRNA designed to downregulate or inhibit the expression of DUX4 to a cell or to a subject in need thereof. In some aspects, the effective dose is therefore a therapeutically effective dose.

In some embodiments, the dose or effective dose of rAAV administered is about 1.0×10¹⁰ vg/kg to about 1.0×10¹⁶ vg/kg. In some aspects, 1.0×10¹⁰ vg/kg is also designated 1.0 E10 vg/kg, which is simply an alternative way of indicating the scientific notation. Likewise, 10¹¹ is equivalent to E11, and the like. In some aspects, the dose of rAAV administered is about 1.0×10¹¹ vg/kg to about 1.0×10¹⁵ vg/kg. In some aspects the dose of rAAV is about 1.0×10¹⁰ vg/kg, about 2.0×10¹⁰ vg/kg, about 3.0×10¹⁰ vg/kg, about 4.0×10¹⁰ vg/kg, about 5.0×10¹⁰ vg/kg, about 6.0×10¹⁰ vg/kg, about 7.0×10¹⁰ vg/kg, about 8.0×10¹⁰ vg/kg, about 9.0×10¹⁰ about 1.0×10¹¹ vg/kg, about 2.0×10¹¹ vg/kg, about 3.0×10¹¹ vg/kg, about 4.0×10¹¹ vg/kg, about 5.0×10¹¹ vg/kg, about 6.0×10¹¹ vg/kg, about 7.0×10¹¹ vg/kg, about 8.0×10¹¹ vg/kg, about 9.0×10¹¹ vg/kg, about 1.0×10¹² vg/kg, about 2.0×12¹² vg/kg, about 3.0×10¹² vg/kg, about 4.0×10¹² vg/kg, about 5.0×10¹² vg/kg, about 6.0×10¹² vg/kg, about 7.0×10¹² vg/kg, about 8.0×10¹² vg/kg, about 9.0×10¹² vg/kg, about 1.0×10¹³ vg/kg, about 2.0×10¹³ vg/kg, about 3.0×10¹³ vg/kg, about 4.0×10¹³ vg/kg, about 5.0×10¹³ vg/kg, about 6.0×10¹³ vg/kg, about 7.0×10¹³ vg/kg, about 8.0×10¹³ vg/kg, about 9.0×10¹³ vg/kg, about 1.0×10¹⁴ vg/kg, about 2.0×10¹⁴ vg/kg, about 3.0×10¹⁴ vg/kg, about 4.0×10¹⁴ vg/kg, about 5.0×10¹⁴ vg/kg, about 6.0×10¹⁴ vg/kg, about 7.0×10¹⁴ vg/kg, about 8.0×10¹⁴ vg/kg, about 9.0×10¹⁴ vg/kg, about 1.0×10¹⁵ vg/kg, about 2.0×10¹⁵ vg/kg, about 3.0×10¹⁵ vg/kg, about 4.0×10¹⁵ vg/kg, about 5.0×10¹⁵ vg/kg, about 6.0×10¹⁵ vg/kg, about 7.0×10¹⁵ vg/kg, about 8.0×10¹⁵ vg/kg, about 9.0×10¹⁵ vg/kg, or about 1.0×10¹⁶ vg/kg.

In some aspects, the dose is about 1.0×10¹¹ vg/kg to about 1.0×10¹⁵ vg/kg. In some aspects, the dose is about 1.0×10¹³ vg/kg to about 5.0×10¹³ vg/kg. In some aspects, the dose is about 2.0×10¹³ vg/kg to about 4.0×10¹³ vg/kg. In some aspects, the dose is about 3.0×10¹³ vg/kg.

In some aspects, an initial dose is followed by a second greater dose. In some aspects, an initial dose is followed by a second same dose. In some aspects, an initial dose is followed by one or more lesser doses. In some aspects, an initial dose is followed by multiple doses which are the same or greater doses.

Methods of transducing a target cell with a delivery vehicle (such as rAAV), in vivo or in vitro, are contemplated. Transduction of cells with an rAAV of the disclosure results in sustained expression of the DUX4 antisense sequence. In some aspects, the disclosure thus provides rAAV and methods of administering/delivering rAAV which express antisense sequence that binds to key exon definition elements in the pre-mRNA, inhibiting the recognition of a specific exon by the spliceosome, leading to exclusion of the target exon from the mature RNA to a subject. In some aspects, the subject is a mammal. In some aspects, the mammal is a human. These methods include transducing cells and tissues (including, but not limited to, tissues such as muscle) with one or more rAAV described herein. Transduction may be carried out with gene cassettes comprising cell-specific control elements. The term “transduction” is used to refer to, as an example, the administration/delivery of u7snRNA comprising antisense sequence, e.g., U7-asDUX4, to a target cell either in vivo or in vitro, via a replication-deficient rAAV described herein resulting in the decreased expression or inhibition of expression of DUX4 by the target cell.

The in vivo methods comprise the step of administering an effective dose, or effective multiple doses, of a composition comprising a delivery vehicle (such as rAAV) to a subject (including a human subject) in need thereof. Thus, methods are provided of administering an effective dose (or doses, administered essentially simultaneously or doses given at intervals) of rAAV described herein to a subject in need thereof. If the dose or doses is administered prior to development of a disorder/disease, the administration is prophylactic. If the dose or doses is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

In some embodiments, compositions and methods of the disclosure are used in treating, ameliorating, or preventing a disease, such as a muscular dystrophy (MD). In various aspects, such MD is FSHD. FSHD is among the most commonly inherited muscular dystrophies, estimated to affect as many as 870,000 individuals. Classical descriptions of FSHD presentation include progressive muscle weakness in the face, shoulder-girdle and arms, but disease can manifest more broadly, including in muscles of the trunk and lower extremities. Variability is also commonly seen within individuals, as asymmetrical weakness is common. Age-at-onset can range from early childhood to adulthood, and is usually related to disease severity, where earlier onset is often associated with more severe muscle weakness. Although most patients with FSHD have a normal life span, respiratory insufficiency can occur, and the disease can be debilitating, as approximately 25% of affected individuals may become wheelchair dependent by their fifties, and even earlier in more severe forms of the disease, while others maintain lifelong ambulation.

FSHD is caused by aberrant expression of the double homeobox 4 gene (DUX4), which produces a transcription factor that is toxic to skeletal muscle. DUX4 is normally functional during the two-cell stage of human development but repressed thereafter in essentially all other tissues, except perhaps the testes. In skeletal muscles of people with FSHD, specific genetic and epigenetic factors conspire to permit DUX4 de-repression, where it then initiates several aberrant gene expression cascades, including those involved in differentiation abnormalities, oxidative stress, inflammatory infiltration, cell death and muscle atrophy.

In families known to carry pathological FSHD, the methods of the disclosure, in various aspects, are methods of preventing disease and they are carried out before the onset of disease. In other various aspects, the methods of the disclosure are carried out after diagnosis and, therefore, are methods of treating or ameliorating disease.

In some embodiments, compositions and methods of the disclosure are used in treating, ameliorating, or preventing a disease, such as a cancer. DUX4 has been shown to be activated in some cancer types, where it functions to mask tumor cells from the immune system (Chew et al., Dev. Cell 2019 Sep 9; 50(5):658-71). For example, DUX4 protein fusions are known to cause cancer, such as rhabdomyosarcoma and Ewing's sarcoma. A CIC-DUX4 gene fusion induces sarcomas and drives sarcoma metastasis (Yoshimoto et al., Cancer Res. 2017 Jun 1; 77(11): 2927-2937; Okimoto et al., J Clin Invest. 2019;129(8): 3401-3406). Thus, the nucleic acids, rAAV and compositions described herein are used in inhibiting DUX4 expression in the treatment, amelioration, or prevention of cancer.

Molecular, biochemical, histological, and functional outcome measures demonstrate the therapeutic efficacy of the products and methods disclosed herein for decreasing the expression of the DUX4 gene and protein and treating muscular dystrophies, such as FSHD. Outcome measures are described, for example, in Chapters 32, 35 and 43 of Dyck and Thomas, Peripheral Neuropathy, Elsevier Saunders, Philadelphia, PA, 4^{th} Edition, Volume 1 (2005) and in Burgess et al., Methods Mol. Biol., 602: 347-393 (2010). Outcome measures include, but are not limited to, reduction or elimination of DUX4 mRNA or protein in affected tissues. The lack of expression of DUX4 and/or the downregulation of expression of DUX4 in the cell is detected by measuring the level of DUX4 protein by methods known in the art including, but not limited to, RT-PCR, QRT-PCR, RNAscope, Western blot, immunofluorescence, or immunohistochemistry in muscle biopsied before and after administration of the rAAV to determine the improvement.

In some embodiments, the level of DUX4 gene expression or protein expression in a cell of the subject is decreased after administration of antisense snRNA construct or the vector, e.g., rAAV, comprising the antisense snRNA construct as compared to the level of DUX4 gene expression or protein expression before administration of the antisense snRNA construct or the vectors, e.g. rAAV. In some aspects, expression of a DUX4 is decreased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%. In various aspects, improved muscle strength, improved muscle function, and/or improved mobility and stamina show an improvement by at least about 2%, at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%.

Other outcome measures include measuring the level of serum creatinine kinase (CK) in the subject before and after treatment. Increased CK levels are a hallmark of muscle damage. In muscular dystrophy patients, CK levels are significantly increased above the normal range (10 to 100 times the normal level since birth). When elevated CK levels are found in a blood sample, it usually means muscle is being disintegrated by some abnormal process, such as a muscular dystrophy or inflammation. Thus, a positive therapeutic outcome for treatment with the methods of the disclosure is a reduction in the level of serum creatinine kinase after administration of the rAAV as compared to the level of serum creatinine kinase before administration of the rAAV.

Other outcome measures include measuring to determine if there is improved muscle strength, improved muscle function, improved mobility, improved stamina, or a combination of two or more thereof in the subject after treatment. Such outcome measures are important in determining muscular dystrophy progression in the subject and are measured by various tests known in the art. Some of these tests include, but are not limited to, the six minute walk test, time to rise test, ascend 4 steps test, ascend and descend 4 steps test, North Star Ambulatory Assessment (NSAA) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

Combination therapies are also included the disclosure. Combination, as used herein, includes both simultaneous treatment and sequential treatments. Combinations of methods described herein with standard medical treatments and supportive care are specifically contemplated, as are combinations with therapies, such as glucocorticoids. All types of glucocorticoids are included for use in the combination therapies disclosed herein. Such glucocorticoids include, but are not limited to, prednisone, prednisolone, dexamethasone, deflazacort, beclomethasone, betamethasone, budesonide, cortisone, hydrocortisone, methylprednisolone, and triamcinolone.

Other combination therapies included in the disclosure are the U7-snRNA, as described herein, in combination with other U7-snRNAs, or in combination with miRNA- based gene therapy, a small molecule inhibitor of DUX4 expression, oligonucleotides to inhibit DUX4 through RNAi or RNAse H or exon skipping mechanisms, U7-snRNA plus a theoretical CRISPR-based gene therapy approach.

Administration of an effective dose of a nucleic acid, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In some aspects, an effective dose is delivered by a systemic route of administration, i.e., systemic administration. Systemic administration is a route of administration into the circulatory system so that the entire body is affected. Such systemic administration, in various aspects, takes place via enteral administration (absorption of the drug through the gastrointestinal tract) or parenteral administration (generally via injection, infusion, or implantation). In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure, in various aspects, are chosen and/or matched by those skilled in the art taking into account the condition or state of the disease or disorder being treated, the condition, state, or age of the subject, and the target cells/tissue(s) that are to express the nucleic acid or protein.

In particular, actual administration of delivery vehicle (such as rAAV) may be accomplished by using any physical method that will transport the delivery vehicle (such as rAAV) into a target cell of an animal. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

“Treating” includes ameliorating or inhibiting one or more symptoms of a muscular dystrophy including, but not limited to, muscle wasting, muscle weakness, myotonia, skeletal muscle problems, abnormalities of the retina, hip weakness, facial weakness, abdominal muscle weakness, joint and spinal abnormalities, lower leg weakness, shoulder weakness, hearing loss, muscle inflammation, and nonsymmetrical weakness.

Administration of an effective dose of a nucleic acid, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In some aspects, an effective dose is delivered by a systemic route of administration, i.e., systemic administration. Systemic administration is a route of administration into the circulatory system so that the entire body is affected. Such systemic administration, in various aspects, takes place via enteral administration (absorption of the drug through the gastrointestinal tract) or parenteral administration (generally via injection, infusion, or implantation). In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure, in various aspects, are chosen and/or matched by those skilled in the art taking into account the condition or state of the disease or disorder being treated, the condition, state, or age of the subject, and the target cells/tissue(s) that are to express the nucleic acid or protein.

In particular, actual administration of delivery vehicle (such as rAAV) may be accomplished by using any physical method that will transport the delivery vehicle (such as rAAV) into a target cell of an animal. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure also provides a kit comprising a nucleic acid, vector, or composition of the disclosure or produced according to a process of the disclosure. In the context of the disclosure, the term “kit” means two or more components, one of which corresponds to a nucleic acid, vector, or composition of the disclosure, and the other which corresponds to a container, recipient, instructions, or otherwise. A kit, therefore, in various aspects, is a set of products that are sufficient to achieve a certain goal, which can be marketed as a single unit.

The kit may comprise one or more recipients (such as vials, ampoules, containers, syringes, bottles, bags) of any appropriate shape, size and material containing the nucleic acid, vector, or composition of the disclosure in an appropriate dosage for administration (see above). The kit may additionally contain directions or instructions for use (e.g. in the form of a leaflet or instruction manual), means for administering the nucleic acid, vector, or composition, such as a syringe, pump, infuser or the like, means for reconstituting the nucleic acid, vector, or composition and/or means for diluting the nucleic acid, vector, or composition.

In some aspects, the kit comprises a label and/or instructions that describes use of the reagents provided in the kit. The kits also optionally comprise catheters, syringes or other delivering devices for the delivery of one or more of the compositions used in the methods described herein.

The disclosure also provides kits for a single dose of administration unit or for multiple doses. In some embodiments, the disclosure provides kits containing single-chambered and multi-chambered pre-filled syringes.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning.

Unless otherwise indicated, the term “at least” preceding a series of elements is to be understood to refer to every element in the series. Those skilled in the art will recognize, or be able to ascertain using no more than routine experimentation, many equivalents to the specific embodiments of the disclosure described herein. Such equivalents are intended to be encompassed by the disclosure.

The term “and/or” wherever used herein includes the meaning of “and”, “or” and “all or any other combination of the elements connected by said term.”

The term “about” or “approximately” as used herein means within 20%, preferably within 10%, and more preferably within 5% of a given value or range. It includes, however, also the concrete number, e.g., about 10 includes 10.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this disclosure is not limited to the particular methodology, protocols, material, reagents, and substances, etc., described herein and as such can vary. The terminology used herein is for the purpose of describing particular embodiments only, and is not intended to limit the scope of the subject matter of the disclosure, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

### Materials and Methods

### Designing DUX4 Targeting U7-snRNAs

The Human Splicing Finder version 3.1 program from Marseille University (http-colon-slash-slash-www.umd.be-slash-HSF-slash) was used to predict potential splice acceptor (SA), splice donor (SD), and splice enhancer sites at the end of DUX4 exon 1 (coding sequence) and within the untranslated exons 1 and 2. For designing U7-snRNAs against DUX4 (called U7-asDUX4s), 18 high-scoring target sites were selected with the fewest number of CpGs and one non-targeting region (Table 1). Predicted off-target matches were determined by BLAST, using each sequence against the human genome database (https colon-slash-slash-blast.ncbi.nlm.nih.gov). The expression cassettes of all U7-asDUX4s, containing a mouse U7 promoter, were synthesized and cloned into pUCIDT plasmid (Integrated DNA Technologies, Coralville, Iowa). Sequences were also designed to bind the DUX4 start codon and poly A signal via reverse complementary base pairing (Table 1). The non-targeting control snRNA antisense sequence is 5′-GTCATGTCGCGTGCCCCGGTGGTCGACACGTCGG-3′ (SEQ ID NO:43).

### Cell Cultures

Human embryonic kidney (HEK293) cells were cultured in DMEM, supplemented with 10% fetal bovine serum, 1% L-glutamine, and 1% penicillin/streptomycin at 37 C in 5% CO₂. Affected and unaffected immortalized human myoblasts derived from a human FSHD patient and an unaffected relative 15Abic and 15Vbic (40,60) were expanded in DMEM media supplemented with 16% Medium 199, 20% fetal bovine serum, 1% penicillin/streptomycin, 30 ng/ml zinc sulphate, 1.4 mg/ml vitamin B12, 55 ng/ml dexamethasone, 2.5 ng/ml human growth factor, 10 ng/ml fibroblast growth factor and 20 mM HEPES. Cells were maintained as myoblasts and differentiated for DUX4 and DUX4-activity biomarker screening by qRT-PCR and RNAscope. To differentiate myoblasts into myotubes, transfected myoblasts were switched to differentiation medium composed of 4:1 ratio of DMEM:Medium 199, supplemented with 15% KnockOut Serum (ThermoFisher Scientific), 2 mM L-glutamine, and 1% antibiotics/antimycobiotics for up to 7 days before harvesting.

### Viability Assay

HEK293 cells (250,000 cells/well) were co-transfected (Lipofectamine-2000, Invitrogen) with an expression plasmid from which full-length DUX4 pre-mRNA (DUX4-fl) was transcribed from the cytomegalovirus (CMV) promoter (CMV.DUX4-fl), along with plasmids expressing either U7-asDUX4 snRNAs or the non-targeting U7-snRNA in a 1:6 ratio using the protocol. The cells were trypsinized at 48 hours. post-transfection and collected in 1 ml of growth media. Automated cell counting was performed using the Countess® Cell Counting Chamber Slides (Thermo Fisher). The results were confirmed with traditional cell counting using a hemacytometer and trypan blue staining. Three independent experiments were performed, and data reported as a mean of total cell number±SEM per group.

### Cell Death Assay

HEK293 cells (42,000 cells/well) were plated on a 96-well plate 16 hours prior to transfection. The next morning, the cells were co-transfected (Lipofectamine-2000, Invitrogen) with CMV.DUX4-fl and U7-asDUX4 snRNAs or a non-targeting U7-snRNA expression plasmid in a 1:6 molar ratio. Cell death was measured using the Apo-ONE Homogeneous Caspase-3/7 Assay (Promega, Madison, WI) at 48 hours post-transfection using a fluorescent plate reader (Spectra Max M2, Molecular Devices, Sunnyvale, CA). Three individual assays were performed in triplicate (n=3), and data averaged per experiment and reported as mean caspase activity±SEM relative to the control assay which was transfected with CMV.DUX4-fl only.

### Western Blot Assay

For this experiment, DUX4 expression plasmids were used, with and without epitope tags (CMV.Myc-DUX4-fl, which contained a myc epitope tag fused to the DUX4 N-terminus; or CMV-DUX4-fl). HEK293 cells were co-transfected in a 1:6 ratio of DUX4:U7asDUX4 expression plasmids. Twenty hours after transfection, cells were lysed in RIPA buffer (50 mM Tris, 150 mM NaCl, 0.1% SDS, 0.5% sodium deoxycholate, 1% Triton X 100), supplemented with a cocktail containing protease inhibitors. Protein concentration was determined by the Lowry protein assay kit (Bio-Rad Laboratories). 25 μg of each total protein sample was run on 12% SDS—polyacrylamide gel. The proteins were transferred to PVDF membranes via a semi-dry transfer method, then blocked in 5% non-fat milk, and incubated with primary monoclonal mouse anti-DUX4 (1:500; P4H2, Novus Biologicals), mouse anti-Myc (R95125, Invitrogen), or rabbit polyclonal anti-α Tubulin antibodies (1:1,000; ab15246, Abcam, Cambridge, MA) overnight at 4° C. The next day, following multiple washes, blots were then probed with horseradish peroxidase (HRP)-conjugated goat anti-mouse or goat anti-rabbit secondary antibodies (1:100,000; Jackson ImmunoResearch, West Grove, PA) for 1 hr. at room temperature. Relative protein bands were developed on X-ray films after short incubation in Immobilon Chemiluminescent HRP Substrate (Millipore, Billerica, MA). Protein quantification was assessed by ImageJ software (National Institutes of Health, Bethesda, Maryland, USA, imagej.nih.gov/ij/).

### Immunofluorescence Staining

Subcellular localization of V5 epitope-tagged DUX4 protein in treated and untreated cells was visualized using V5 immunofluorescence staining (Wallace et al. (2011) Ann Neurol 69, 540-552. This plasmid carried a full-length DUX4 sequence consisting of the coding and 3′ UTR sequences but engineered to express DUX4 protein with an in-frame carboxy-terminal V5 epitope fusion. Twenty hours after transfection, the cells were fixed in 4% paraformaldehyde (PFA) for 20 minutes, and nonspecific antigens were blocked with 5% BSA in PBS, supplemented with 0.2% Triton X-100. The cells were incubated at 4° C., overnight, in rabbit polyclonal anti-V5 primary antibody (1:2,500; Abcam, ab9116). The following day, cells were washed with PBS, incubated with goat anti-rabbit Alexa-594 secondary antibodies (1:2,500; Invitrogen), and mounted with Vectashield mounting medium containing DAPI (Vector Laboratories, Burlingame, CA).

### RNAscope Assay and Quantification

Detecting over-expressed DUX4 in HEK293s. RNAscope in situ hybridization assay was used to measure DUX4 mRNA levels following co-transfection of CMV.DUX4-fl and U7.asDUX4 expression plasmids in HEK293 cells (1:6 ratio). Specifically, HEK293 cells were seeded in triplicate on glass coverslips in 24-well plates at a density of 120,000 cells per well, 16 hours prior transfection. The next morning, upon reaching 70% confluency, cells were cotransfected with 250 ng of CMV.DUX4-fl expression plasmid (Lipofectamine-2000, Thermo Fisher Scientific), according to manufacturer's instructions. Sixteen hours after transfection, cells were fixed with 4% PFA and RNAscope staining was performed following manufacturer's instructions (detailed below).

Detecting endogenous DUX4 in human FSHD myotubes. To determine the specificity of U7-asDUX4 snRNAs for targeting endogenous DUX4 mRNA, 15Abic FSHD myoblasts (15A, 500,000 cells/reaction) were transfected with U7-asDUX4 expression plasmids via electroporation (Lonza, VVPD-1001) and then differentiated into myotubes for up to 7 days. RNAscope staining was performed as described previously (Amini Chermahini, et al. (2019) RNA 25, 1211-1217). The cells were fixed in 4% PFA and dehydrated/rehydrated with ethanol gradients. Endogenous peroxidase activity was blocked by hydrogen peroxide treatment. Protease III was added to increase the permeability of fixed cells for RNAscope probes. The cells were treated with a DUX4-specific RNAscope probe (ACDBio, Cat. No. 498541) or probes to detect the positive control housekeeping peptidylprolyl isomerase B (PPIB) and negative control bacterial gene, dihydrodipicolinate reductase (dapB). Following probe incubation, cells were treated with several signal amplification steps using RNAscope 2.5 HD Assay Brown, according to the manufacturer's protocol (ACDBio). The cells were counterstained with 50% Gill's hematoxylin I (cat. No. HXGHE1 LT, American Master Tech Scientific) for 2 min at room temperature, followed by several washes. After mounting, images were captured using an Olympus DP71 microscope. DUX4 RNAscope signals were quantified using ImageJ-Fiji software as described previously (30).

### Quantitative Real Time-PCR Analysis of DUX4 Biomarkers

15A FSHD myoblasts were transfected as described herein in the RNAscope section and differentiated into myotubes. The total RNA content was extracted using TRIzol Reagent (ThermoFisher) according to the manufacturer's protocol, and yield measured by Nanodrop. Isolated RNA was then Dnase-treated (DNA-Free, Ambion, TX), and cDNA was generated with the High-Capacity cDNA Reverse Transcription Kit (Applied Biosystems) using random hexamer primers. Subsequent cDNA samples were then used as a template for the Taqman Assay using pre-designed TRIM43, MBD3L2, PRAMEF12, ZSCAN4 (biomarkers of DUX4 activity), and human RPL13A control primer/probe sets (Applied Biosystems). All data were normalized to the non-targeting-U7-snRNA transfected cells. Data were generated from two independent experiments performed in triplicate for each biomarker.

### Statistical Analysis

All statistical analyses (Caspase 3/7 assay, Cell Viability Assay, RNAscope quantification, Western blot, qRT-PCR) were performed in Graph Pad Prism 5 (Graph Pad Software, La Jolla, CA) using the indicated statistical tests.

### Example 2

### DUX4-Targeting U7-snRNAs Reduce Apoptosis and Increase the Viability of Co-Transfected HEK293s

Recombinant U7-snRNAs were previously developed to induce skipping of mutated exons as potential treatment for Duchenne Muscular Dystrophy (Goyenvalle et al. (2012) Mol Ther 20, 1212-1221) and β-thalassaemia (Nualkaew et al. (2019) Sci Rep 9, 7672). In these studies, U7-snRNAs were used to restore the expression of frame-shifted genes by skipping entire exons. In contrast, the goal of this study was to develop a novel gene silencing strategy by using U7-snRNAs to interfere with DUX4 pre-mRNA maturation or to inhibit translational initiation (Biferi et al. (2017) Mol Ther 25, 2038-2052; Wein et al. (2014) Nat Med 20, 992-1000).

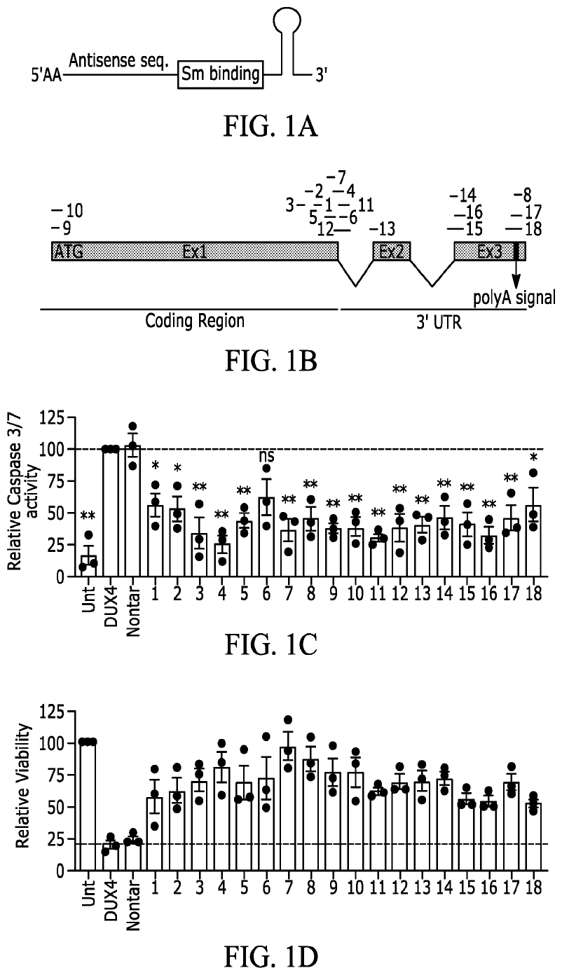

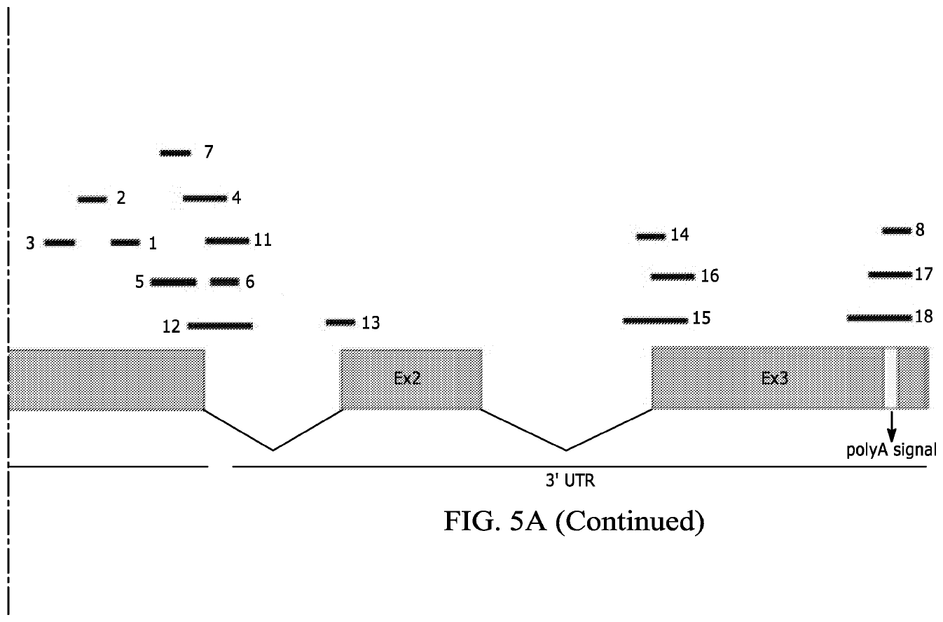

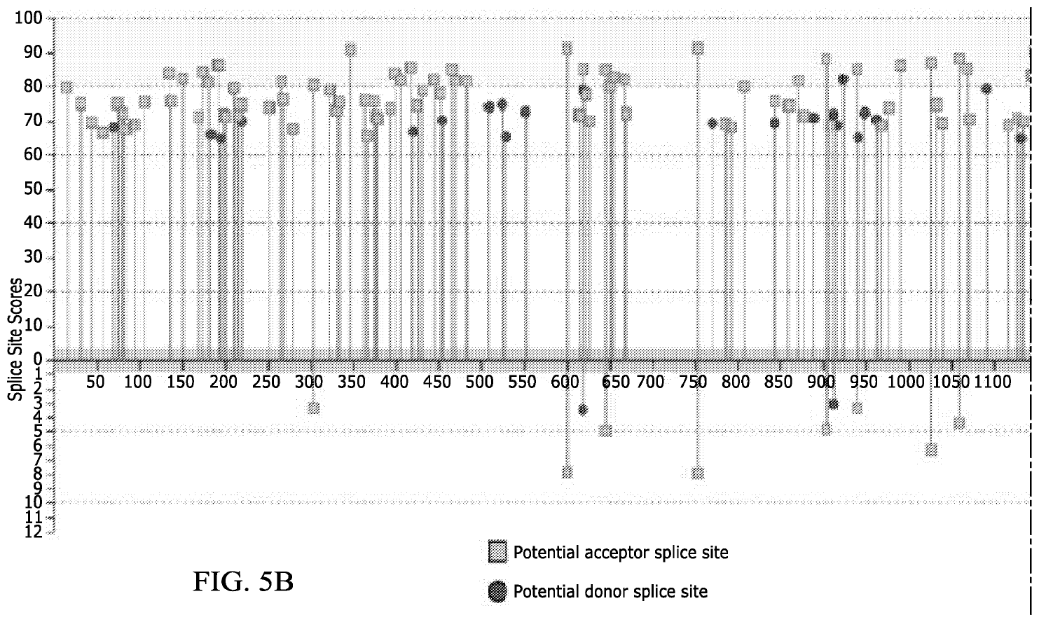

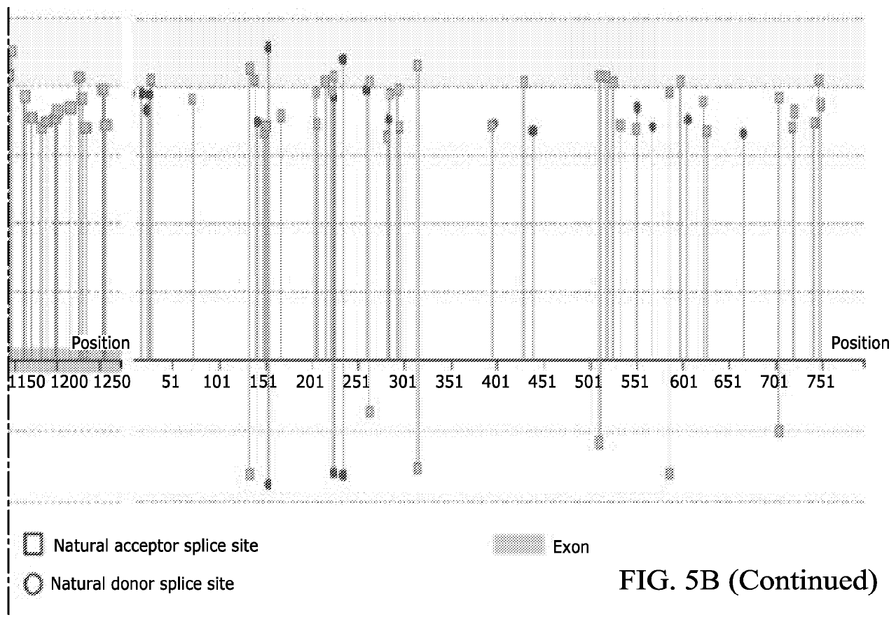

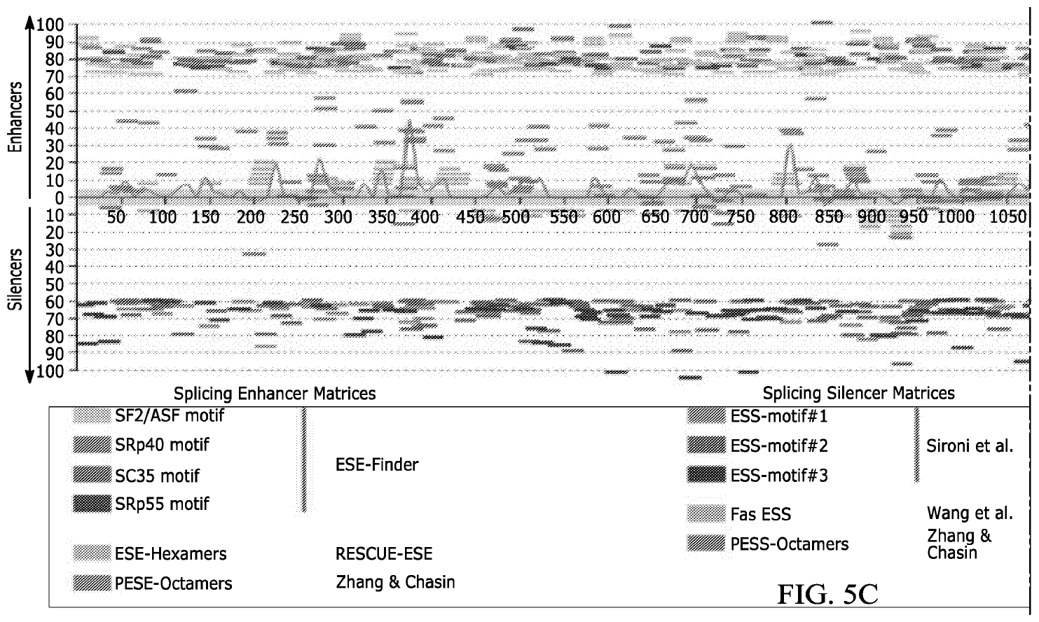

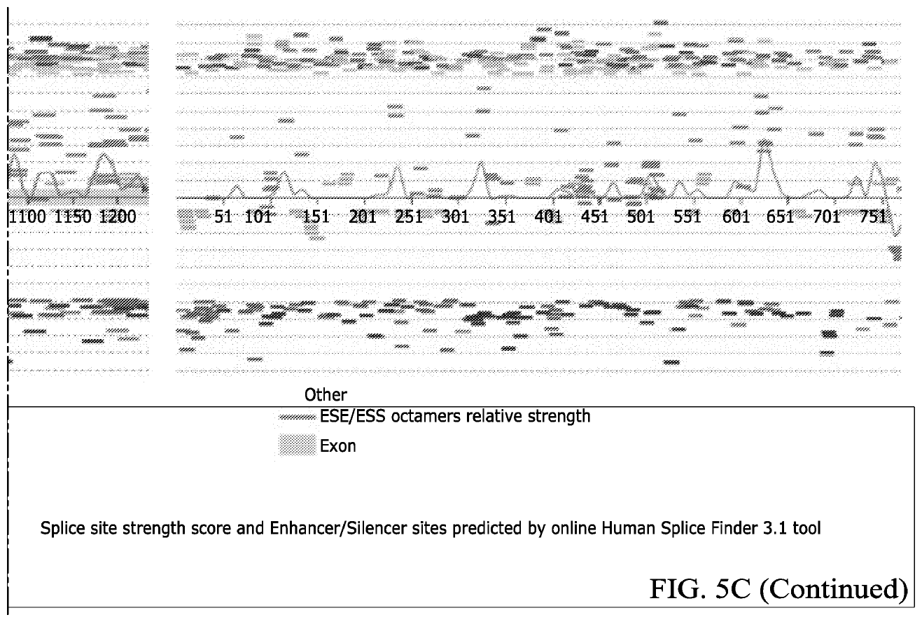

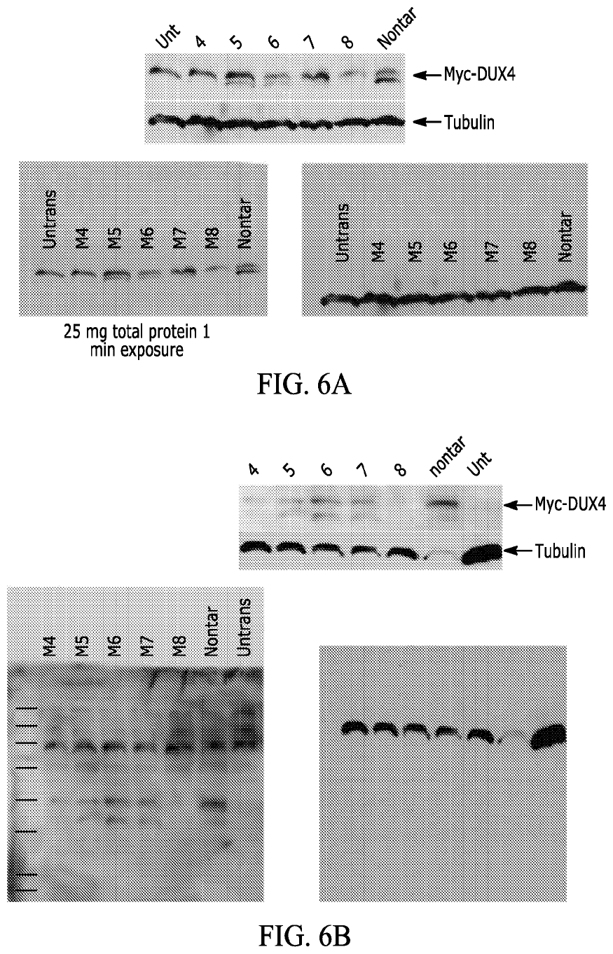

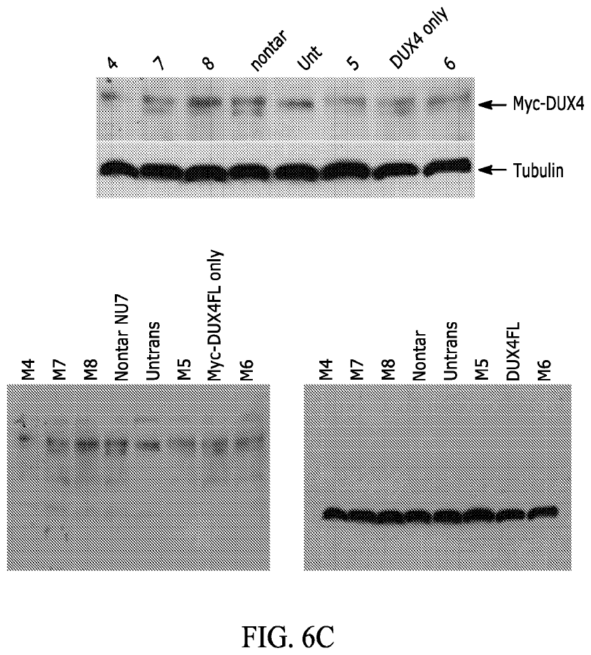

To do this, recombinant U7-snRNAs targeting splice donor (SD), splice acceptor (SA) and splice enhancer (SE) sequences, or the polyadenylation signal (PAS) in DUX4 exon 3 were developed (FIG. 1A-B). In addition, two constructs (9 and 10), designed to sit atop the full-length DUX4 start codon and potentially interfere with translation, were generated. The structure of these DUX4-targeting U7-snRNAs (called U7-asDUX4) is shown in FIG. 1A, wherein a key feature for specificity is an antisense sequence modified to base pair with various regions of the DUX4 pre-mRNA. To choose effective sequences for interfering with correct splicing, the Human Splicing Finder tool (FIG. 5A-C) was used to predict potential SD, SA, and SE sites for all three DUX4 exons, and within introns 1 and 2. U7-asDUX4s were then designed to target the highest-scoring sites (FIG. 1B). For those U7-asDUX4s targeting the polyA signal or start codon, it was ensured that antisense sequences provided complete coverage of the cognate sites on the DUX4 mRNA. All U7-asDUX4 sequences and their important features are summarized in Tables 1 and 2 herein.

HEK293 cells do not normally express detectable DUX4 but are susceptible to DUX4-induced cell death following transfection with a CMV.DUX4 expression plasmid. Therefore, the efficacy of U7-asDUX4 expression plasmids was initially assessed by measuring apoptotic cell death using Caspase-3/7 and cell viability assays as outcome measures in co-transfected HEK293 cells. 18 U7-asDUX4 sequences were designed, and the constructs were made and tested. Of these 18 constructs, 13 significantly reduced cell death (50%) and increased viability (50%) of co-transfected HEK293 cells (FIG. 1C-D). The most effective U7-asDUX4s were constructs 4, 7, and 8, targeting the exon 1-intron 1 junction or the polyA signal (PAS). These constructs reduced Caspase-3/7 activity by 75%±7, 60%±9, and 50%±8 respectively, and increased viability significantly more than other U7-asDUX4s by 78.5%±8.4, 94.8%±4.9, and 84.8%±3.8 respectively, compared to only DUX4 (19.1%±1.6) or DUX4 with nontarget U7-snRNA (23.3%±0.5) (FIG. 1C-D). Due to their superior protective properties, U7-asDUX4 constructs 4, 7 and 8 appeared to be leading candidate sequences.

### Example 3

### U7-asDUX4s Significantly Decrease DUX4 Expression in Transfected HEK293 Cells

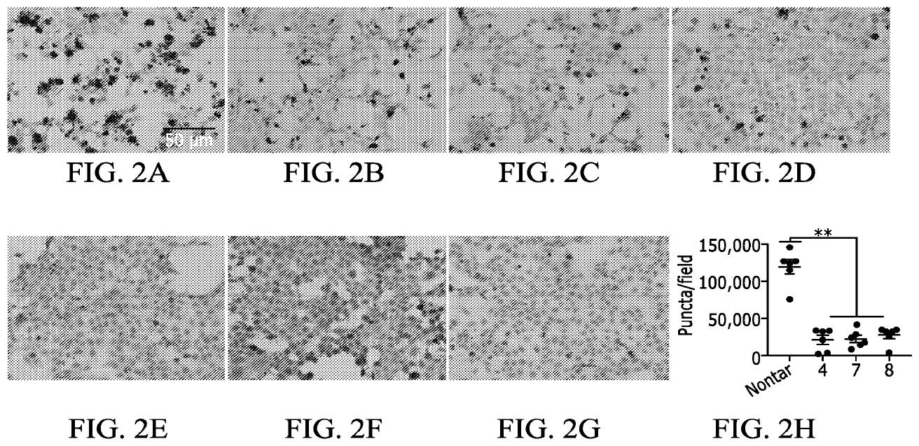

The reduction in DUX4-related cell death outcomes in samples treated with U7-asDUX4 plasmids suggested these sequences operated to inhibit full-length DUX4 gene expression. To investigate the specificity of suspected lead U7-asDUX4 sequences to target and reduce overexpressed DUX4 mRNA in HEK293 cells, an RNAscope in situ hybridization assay was used first to detect DUX4 mRNA in co-transfected cells. Fixed cells were incubated with probes targeting DUX4, control transcripts, or negative control reagents, then treated with a diaminobenzidine (DAB) reagent that stains hybridized target mRNAs brown. As previously reported (Amini Chermahini et al. (2019) RNA 25, 1211-1217), cells transfected with DUX4 expression plasmid alone showed abundant, spider-like brown signals when incubated with DUX4 probes, as well as relatively low cell density consistent with death (FIG. 2A). In contrast, DUX4 probe signal was significantly reduced in cells co-transfected with DUX4 and our three lead U7-asDUX4 (FIG. 2B-D). Specifically, in U7-asDUX4-treated wells, there were significantly fewer DUX4-positive cells and/or reduced intensity of DUX4 staining in cells that still showed DUX4 signal (FIG. 2B-D). Positive and negative controls behaved as expected, since no DUX4 signal in un-transfected HEK293 cells was found (FIG. 2E), while abundant signal was evident in cells stained with probes to the peptidylprolyl isomerase B (PPIB) gene (FIG. 2F), a positive control for the RNAscope assay. Consistent with DUX4 knockdown that provided some protection from cell death (FIG. 1D), wells transfected with U7-asDUX4 plasmids had greater cell density compared to “DUX4-only” transfected samples.

### Example 4

### U7-asDUX4 snRNAs Reduce Full-Length DUX4 Protein in Transfected HEK293 Cells

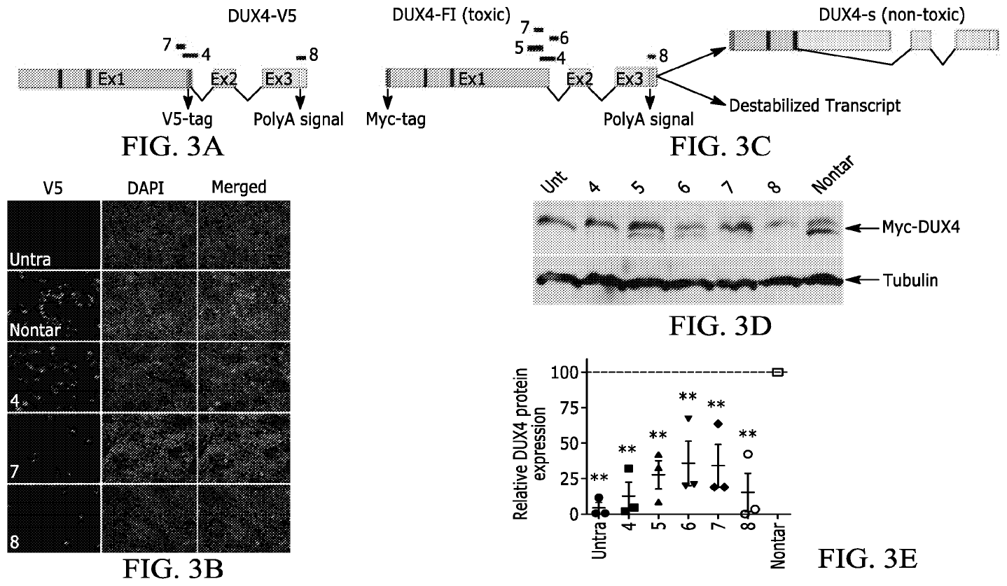

To confirm full-length DUX4 mRNA knockdown, the efficiency of U7-asDUX4 to suppress DUX4 protein expression was determined. To do this, cells were co-transfected cells with three lead U7-asDUX4 constructs, or a non-targeting control, along with a CMV.DUX4 expression plasmid. To facilitate protein detection, a full-length DUX4 construct containing an in-frame COOH-terminal fusion of the V5 epitope tag (CMV.DUX4.V5-fl) was used (FIG. 3A). To detect DUX4 protein, cells were stained with fluorescence-labeled antibodies to the V5 tag. A reduced DUX4.V5 signal was observed in cells co-transfected with U7-asDUX4 sequence 7 or 8, compared to cells that received a non-targeting control U7-snRNA (FIG. 3B). Interestingly, U7-asDUX4-4 did not impact DUX4.V5 protein levels, as its binding site was disrupted by the V5-epitope tag, thereby serving as an inadvertent control for specificity.

To additionally confirm DUX4 protein knockdown by lead U7-snRNAs, similar co-transfection experiments were carried out in HEK293 cells; however, western blots were used to measure outcome. In addition, because the C-terminal V5 tag disrupted the U7-asDUX4-4 binding site, a different DUX4 expression construct was utilized in this set of experiments. Specifically, a CMV.DUX4 expression plasmid containing an amino-terminal, in-frame myc-epitope tag was generated (FIG. 3C). It was reasoned that this construct would allow us to determine if the U7-asDUX4 sequences designed to mask the full-length DUX4 mRNA SD/SA near the 3′ end of exon 1 would bias splicing to produce a truncated and non-toxic DUX4-s protein isoform (FIG. 3C) (Snider et al. (2010) PLoS Genet 6, e1001181). Western blotting was carried out using protein extracts from HEK293 cells co-transfected with plasmids expressing Myc.DUX4 and five different U7-asDUX4 constructs, including 3 leading constructs and sequences 5 and 6, which were designed to base pair near the exon 1/intron 1junction. Using a myc-epitope antibody, the full-length Myc-DUX4 protein band (52 kDa) was detected in all transfected cells. Additionally, all samples contained a larger non-DUX4 protein that migrated at the size of endogenous c-myc protein (˜60 kDa). Consistent with previous experiments, leading constructs, U7-asDUX4 sequences 4, 7 and 8, significantly reduced DUX4 protein by 87.4%±9.8% SEM, 65.9%±15% SEM, and 84.7%±13.5% SEM (n=3 independent experiments; FIGS. 3D-E and 6A-B). No evidence of DUX4-s production was detected by western blot (predicted size 22 kDa), suggesting the reduction of full-length DUX4 gene expression by U7-asDUX4 sequences designed to mask DUX4 splice sites (4, 5, 6 and 7) did not operate by shifting splicing patterns to favor the DUX4-s isoform. Similarly, no evidence of a shorter DUX4-s transcript was found using 3′ RACE RT-PCR (FIG. 7). The non-specific upper band on these western blots showed variable expression. Because DUX4 has been shown to activate Myc, it is possible that changing DUX4 levels could impact the abundance of that upper band, if it is Myc (Shadle et al. (2017) PLoS Genet 13, e1006658). However, a strong correlation between residual DUX4 and Myc abundance in these experiments was not observed.

### Example 5

### U7-asDUX4s Significantly Decrease Endogenous DUX4 Expression in Myotubes from FSHD Patients

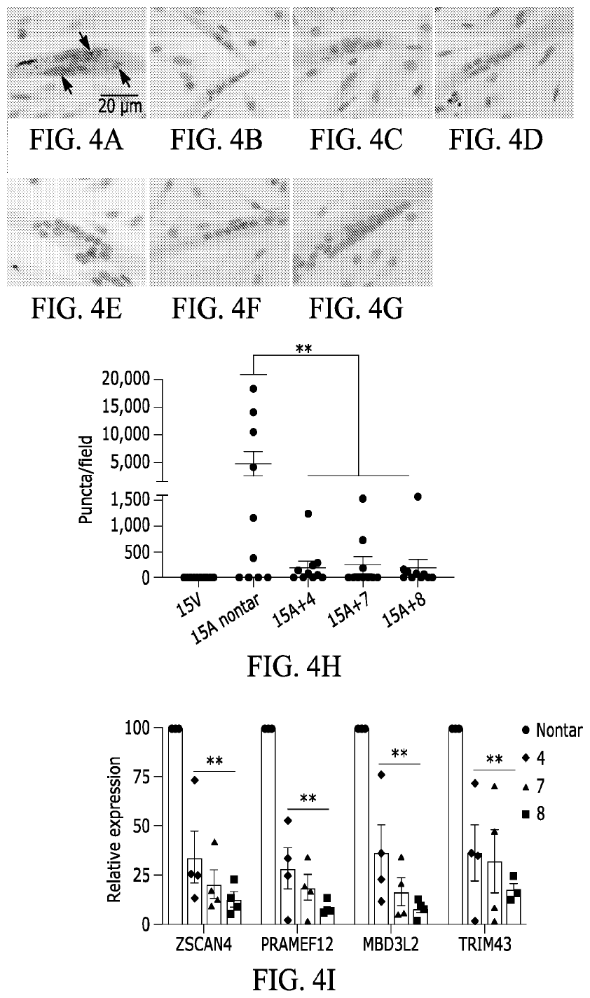

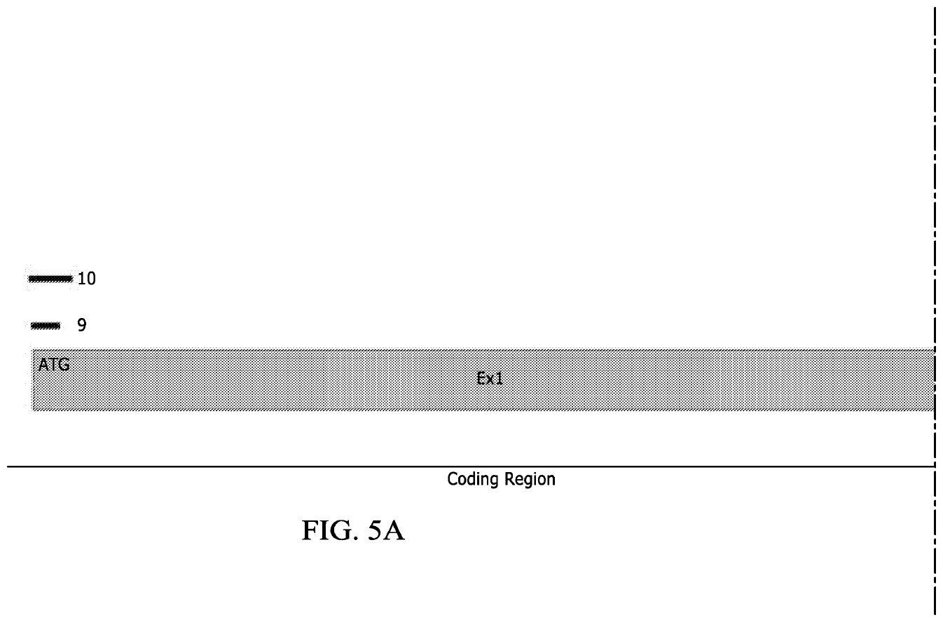

Results from studies in HEK293s suggested that several U7-asDUX4 snRNAs could reduce full-length DUX4 expression and offer protection from cell death in an over-expression model. Thus, the ability of leading U7-asDUX4 snRNA constructs (4, 7, and 8) to decrease endogenous DUX4 mRNA in FSHD patient myotubes was assessed using RNAscope in situ hybridization. RNAscope has previously been used to detect DUX4 in FSHD myotubes (Amini Chermahini et al. (2019) RNA 25, 1211-1217). Consistent with prior reports, it was found that DUX4 staining was only present in a small percentage of cells at any given time. Importantly, DUX4 knockdown following delivery of an artificial DUX4-targeted microRNA (mi405) was able to be quantified (Amini Chermahini et al. (2019) supra; Jones et al. (2012) Hum Mol Genet 21, 4419-4430). RNAscope was therefore used to determine if U7-asDUX4 snRNAs could reduce endogenous DUX4 signal in myotubes derived from human FSHD patients, thereby supporting the potential translatability of this approach. To do this, electroporation was used to transfect FSHD muscle cells, which typically yields ˜50-70% transfection efficiency (FIG. 8). Consistent with previous results, untransfected 15A FSHD myotubes showed brown DUX4 signals, while those transfected with U7-asDUX4 sequences 4, 7, and 8 had significantly reduced RNAscope signals (FIG. 4A-H). These results confirm that these three U7-asDUX4 constructs (i.e., U7-asDUX4 sequences 4, 7, and 8) can effect destabilization and degradation of endogenous DUX4 mRNA in FSHD muscle cells.

### Example 6

### U7-asDUX4 snRNAs Decrease DUX4-Activated Biomarker Expression in FSHD Myotubes

With the emergence of prospective FSHD therapies came a need in the FSHD field to develop clinical outcome measures and biomarkers that could be used to establish therapeutic efficacy (LoRusso et al. (2019) BMC Neurol 19, 224; Mul et al. (2017). Neurology 89, 2057-2065; Wang et al. (2019) Hum Mol Genet 28, 476-486; Wong et al. (2020) Hum Mol Genet 29, 1030-1043). Although DUX4 expression is the most direct measure of target engagement by a prospective drug or gene therapy, it is difficult to detect and relatively scarce in FSHD muscle biopsies. Thus, DUX4 expression in human muscle biopsies is currently not a reliable outcome measure for FSHD clinical trials, and several groups have now turned to examining DUX4-activated biomarkers as an indirect measure of DUX4 expression. At least 67 different genes contain regulatory regions with DUX4 binding sites and are consistently activated upon DUX4 expression. Recent studies suggest, however, that only a small number of biomarkers are needed to represent the entire set (Rickard et al. (2015) Hum Mol Genet 24, 5901-5914; Yao et al. (2014) Hum Mol Genet 23, 5342-5352; Eidahl (2016) Hum Mol Genet 25, 4577-4589; Geng et al. (2012) Dev Cell 22, 38-51; Jagannathan (2016) Hum Mol Genet 25, 4419-4431; van den Heuvel et al. (2019) Hum Mol Genet 28, 1064-1075). Thus, four biomarkers, i.e., ZSCAN4, PRAMEF12, TRIM43, and MBD3L2, were selected in this study because they are established DUX4 target genes and FSHD disease biomarkers and they have consistently shown differential expression between FSHD and healthy control cells (Yao et al. (2014) Hum Mol Genet 23, 5342-5352; Eidahl (2016) Hum Mol Genet 25, 4577-4589; Chen et al. (2016) Mol Ther 24, 1405-1411; Lek et al. (2020) Sci Trans! Med 12.; Amini Chermahini (2019) RNA 25, 1211-1217.). Therefore, the ability of several U7-asDUX4 snRNAs to suppress these DUX4-activated biomarkers in FSHD patient cells was tested.

To do this, 15A FSHD patient myoblasts were transfected with U7-asDUX4 snRNA-4, -7, and -8, as well as a non-targeting control. Cells were then differentiated into myotubes for 7 days and quantitative RT-PCR was carried out to measure the expression of the DUX4-activated human biomarkers TRIM43, MBD3L2, PRAMEF12, and ZSCAN4. The expression of all four biomarkers was present in untreated 15A myotubes and was significantly reduced in U7-asDUX4-treated 15A cells (FIG. 4I).

Several U7-asDUX4 constructs that significantly reduced DUX4 and DUX4-associated outcomes in both co-transfected cells and FSHD patient-derived myotubes were identified. These experiments provide proof-of-concept for DUX4 silencing using novel recombinant U7-asDUX4 as a new approach for the treatment of FSHD. Translating this approach in vivo in FSHD mouse models is being carried out.

### Example 7

### U7-asDUX4 snRNAs Decrease DUX4-Activated Biomarker Expression in a Mouse Model of FSHD

AAV.U7-asDUX4 of the disclosure are injected into a new FSHD mouse model, e.g., a Tamoxifen-Inducible DUX4 (TIC-DUX4) mouse model of severe FSHD (Giesige et al., JCI Insight. 2018; 3(22)) or any other mouse model of FSHD mice intramuscularly (IM) or intravenously (IV). Animals receive 3×10E14 vg/kg, 8×10E13 vg/kg or 3×10E13 vg/kg particles of AAV6 vectors carrying mi405, mi405H, or miLacZ sequences, or saline, via tail vein or intramuscularly.

Animals are randomized and injected in a blinded fashion, thereby requiring two operators: one to set up the blind and dilute vectors for delivery, and a second to inject animals and perform outcome measures. Based on power analysis done in the published characterization of TIC-DUX4 mice, N=20 animals per group provided sufficient power to test outcome measures. However, like humans, TIC-DUX4 mice can show variable phenotypes, so to ensure highly powered studies and protect for attrition, N is increased to 26 (N=13 males and 13 females), for each dose and time point.

In some studies, animals are randomly divided into two groups as follows:

Acute FSHD model. TIC-DUX4 mice or WT mice, treated with Tamoxifen (5 mg/kg 1× per week for 10 weeks) or sunflower oil vehicle via oral gavage.

Chronic FSHD model. TIC-DUX4 mice or WT mice remain uninduced and are allowed to age for 9 months prior to performing outcome measures.

After 4, 8, 12, 16, 20, and 24 weeks, the expression level of a DUX4 biomarker, such as Wfdc3 or Trim36, are measured by qRT-PCR, RNAscope, or ddPCR.

Reduced levels of DUX4 biomarker expression are observed in muscles of mice treated with U7-asDUX4 compared to the levels in muscles of untreated mice.

### Example 8

### U7-asDUX4 snRNAs Decrease Endogenous DUX4 Expression in Muscle in a Mouse Model of FSHD

AAV.U7-asDUX4 of the disclosure are injected into a new FSHD mouse model (TIC-DUX4) or any other mouse model of FSHD mice intramuscularly (IM) or intravenously (IV). After 4, 8, 12, 16, 20, and 24 weeks, the expression level of DUX4 mRNA is measured by qRT-PCR, RNAscope, or ddPCR. For histopathology and molecular analyses, several muscles are collected, including tibialis anterior, gastrocnemius, triceps, biceps, quadriceps, and diaphragm. Absolute and specific force measurements are carried out in gastrocnemius muscles. Because force measurements can negatively impact the integrity of muscle histopathology (i.e. render them non-publication quality for imaging), some extra mice per group are optionally injected to ensure quality images for histopathological analyses.

Reduced levels of DUX4 mRNA are observed in muscles of mice treated with U7-asDUX4 compared to the levels in muscles of untreated mice.

### Example 9

### U7-asDUX4 snRNAs Decrease Endogenous DUX4 Expression in Muscle

AAV.U7-asDUX4 of the disclosure are injected into patients suffering from FSHD intramuscularly (IM) or intravenously (IV). Prior to treatment and after 4, 8, 12, 16, 20, 24, 28, 32, 36 40, 44, 48, and 52 weeks, the expression level of DUX4 mRNA in muscle of the patients is measured in biopsied muscle by qRT-PCR, RNAscope, or ddPCR.

Reduced levels of DUX4 mRNA are observed in muscles of patients treated with U7-asDUX4 compared to the levels of DUX4 mRNA in muscles of the same patients prior to treatment. Improvement in FSHD disease symptoms is also observed.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control. References referred to herein with numbering are provided with the full citation as shown herein below.

### REFERENCES

1. Deenen, J. C., Arnts, H., van der Maarel, S. M., Padberg, G. W., Verschuuren, J. J., Bakker, E., Weinreich, S. S., Verbeek, A. L., and van Engelen, B. G. (2014). Population-based incidence and prevalence of facioscapulohumeral dystrophy. Neurology 83, 1056-1059.
2. Pastorello, E., Cao, M., and Trevisan, C. P. (2012). Atypical onset in a series of 122 cases with FacioScapuloHumeral Muscular Dystrophy. Clin Neurol Neurosurg 114, 230-234.
3. Klinge, L., Eagle, M., Haggerty, I. D., Roberts, C. E., Straub, V., and Bushby, K. M. (2006). Severe phenotype in infantile facioscapulohumeral muscular dystrophy. Neuromuscul Disord 16, 553-558.
4. Statland, J. M., and Tawil, R. (2016). Facioscapulohumeral Muscular Dystrophy. Continuum (Minneap Minn) 22, 1916-1931.
5. Lemmers, R. J., van der Vliet, P. J., Klooster, R., Sacconi, S., Camano, P., Dauwerse, J. G., Snider, L., Straasheijm, K. R., van Ommen, G. J., Padberg, G. W., et al. (2010). A unifying genetic model for facioscapulohumeral muscular dystrophy. Science 329, 1650-1653.
6. Hendrickson, P. G., Dorais, J. A., Grow, E. J., Whiddon, J. L., Lim, J. W., Wike, C. L., Weaver, B. D., Pflueger, C., Emery, B. R., Wilcox, A. L., et al. (2017). Conserved roles of mouse DUX and human DUX4 in activating cleavage-stage genes and MERVL/HERVL retrotransposons. Nat Genet 49, 925-934.
7. Whiddon, J. L., Langford, A. T., Wong, C. J., Zhong, J. W., and Tapscott, S. J. (2017). Conservation and innovation in the DUX4-family gene network. Nat Genet 49, 935-940.
8. Bosnakovski, D., Xu, Z., Gang, E. J., Galindo, C. L., Liu, M., Simsek, T., Garner, H. R., Agha-Mohammadi, S., Tassin, A., Coppee, F., et al. (2008). An isogenetic myoblast expression screen identifies DUX4-mediated FSHD-associated molecular pathologies. EMBO J 27, 2766-2779.
9. Dixit, M., Ansseau, E., Tassin, A., Winokur, S., Shi, R., Qian, H., Sauvage, S., Matteotti, C., van Acker, A. M., Leo, O., et al. (2007). DUX4, a candidate gene of facioscapulohumeral muscular dystrophy, encodes a transcriptional activator of PITX1. Proc Natl Acad Sci U S A 104, 18157-18162.
10. Wallace, L. M., Garwick, S. E., Mei, W., Belayew, A., Coppee, F., Ladner, K. J., Guttridge, D., Yang, J., and Harper, S. Q. (2011). DUX4, a candidate gene for facioscapulohumeral muscular dystrophy, causes p53-dependent myopathy in vivo. Ann Neurol 69, 540-552.
11. Giesige, C. R., Wallace, L. M., Heller, K. N., Eidahl, J. O., Saad, N. Y., Fowler, A. M., Pyne, N. K., Al-Kharsan, M., Rashnonejad, A., Chermahini, G. A., et al. (2018). AAV-mediated follistatin gene therapy improves functional outcomes in the TIC-DUX4 mouse model of FSHD. JCI Insight 3.
12. Kowaljow, V., Marcowycz, A., Ansseau, E., Conde, C. B., Sauvage, S., Matteotti, C., Arias, C., Corona, E. D., Nunez, N. G., Leo, O., et al. (2007). The DUX4 gene at the FSHD1A locus encodes a pro-apoptotic protein. Neuromuscul Disord 17, 611-623.
13. Snider, L., Asawachaicharn, A., Tyler, A. E., Geng, L. N., Petek, L. M., Mayes, L., Miller, D. G., Lemmers, R. J., Winokur, S. T., Tawil, R., et al. (2009). RNA transcripts, miRNA-sized fragments and proteins produced from D4Z4 units: new candidates for the pathophysiology of facioscapulohumeral dystrophy. Hum Mol Genet 18, 2414-2430.
14. Snider, L., Geng, L. N., Lemmers, R. J., Kyba, M., Ware, C. B., Nelson, A. M., Tawil, R., Filippova, G. N., van der Maarel, S. M., Tapscott, S. J., et al. (2010). Facioscapulohumeral dystrophy: incomplete suppression of a retrotransposed gene. PLoS Genet 6, e1001181.
15. Shadle, S. C., Zhong, J. W., Campbell, A. E., Conerly, M. L., Jagannathan, S., Wong, C. J., Morello, T. D., van der Maarel, S. M., and Tapscott, S. J. (2017). DUX4-induced dsRNA and MYC mRNA stabilization activate apoptotic pathways in human cell models of facioscapulohumeral dystrophy. PLoS Genet 13, e1006658.
16. Dmitriev, P., Bou Saada, Y., Dib, C., Ansseau, E., Barat, A., Hamade, A., Dessen, P., Robert, T., Lazar, V., Louzada, R. A. N., et al. (2016). DUX4-induced constitutive DNA damage and oxidative stress contribute to aberrant differentiation of myoblasts from FSHD patients. Free Radic Biol Med 99, 244-258.
17. Rickard, A. M., Petek, L. M., and Miller, D. G. (2015). Endogenous DUX4 expression in FSHD myotubes is sufficient to cause cell death and disrupts RNA splicing and cell migration pathways. Hum Mol Genet 24, 5901-5914.
18. Vanderplanck, C., Ansseau, E., Charron, S., Stricwant, N., Tassin, A., Laoudj-Chenivesse, D., Wilton, S. D., Coppee, F., and Belayew, A. (2011). The FSHD atrophic myotube phenotype is caused by DUX4 expression. PLoS One 6, e26820.
19. Yao, Z., Snider, L., Balog, J., Lemmers, R. J., Van Der Maarel, S. M., Tawil, R., and Tapscott, S. J. (2014). DUX4-induced gene expression is the major molecular signature in FSHD skeletal muscle. Hum Mol Genet 23, 5342-5352.
Eidahl, J. O., Giesige, C. R., Domire, J. S., Wallace, L. M., Fowler, A. M., Guckes, S. M., Garwick-Coppens, S. E., Labhart, P., and Harper, S. Q. (2016). Mouse Dux is myotoxic and shares partial functional homology with its human paralog DUX4. Hum Mol Genet 25, 4577-4589.
21. Lyle, R., Wright, T. J., Clark, L. N., and Hewitt, J. E. (1995). The FSHD-associated repeat, D4Z4, is a member of a dispersed family of homeobox-containing repeats, subsets of which are clustered on the short arms of the acrocentric chromosomes. Genomics 28, 389-397.
22. Gabriels, J., Beckers, M. C., Ding, H., De Vriese, A., Plaisance, S., van der Maarel, S. M., Padberg, G. W., Frants, R. R., Hewitt, J. E., Collen, D., et al. (1999). Nucleotide sequence of the partially deleted D4Z4 locus in a patient with FSHD identifies a putative gene within each 3.3 kb element. Gene 236, 25-32.
23. Marsollier, A. C., Ciszewski, L., Mariot, V., Popplewell, L., Voit, T., Dickson, G., and Dumonceaux, J. (2016). Antisense targeting of 3′ end elements involved in DUX4 mRNA processing is an efficient therapeutic strategy for facioscapulohumeral dystrophy: a new gene-silencing approach. Hum Mol Genet 25, 1468-1478.
24. Chen, J. C., King, O. D., Zhang, Y., Clayton, N. P., Spencer, C., Wentworth, B. M., Emerson, C. P., Jr., and Wagner, K. R. (2016). Morpholino-mediated Knockdown of DUX4 Toward Facioscapulohumeral Muscular Dystrophy Therapeutics. Mol Ther 24, 1405-1411.
25. Ansseau, E., Vanderplanck, C., Wauters, A., Harper, S. Q., Coppee, F., and Belayew, A. (2017). Antisense Oligonucleotides Used to Target the DUX4 mRNA as Therapeutic Approaches in FaciosScapuloHumeral Muscular Dystrophy (FSHD). Genes (Basel) 8.
26. Lek, A., Zhang, Y., Woodman, K. G., Huang, S., DeSimone, A. M., Cohen, J., Ho, V., Conner, J., Mead, L., Kodani, A., et al. (2020). Applying genome-wide CRISPR-Cas9 screens for therapeutic discovery in facioscapulohumeral muscular dystrophy. Sci Transl Med 12.
27. Himeda, C. L., Jones, T. I., and Jones, P. L. (2016). CRISPR/dCas9-mediated Transcriptional Inhibition Ameliorates the Epigenetic Dysregulation at D4Z4 and Represses DUX4-fl in FSH Muscular Dystrophy. Mol Ther 24, 527-535.
28. DeSimone, A. M., Leszyk, J., Wagner, K., and Emerson, C. P., Jr. (2019). Identification of the hyaluronic acid pathway as a therapeutic target for facioscapulohumeral muscular dystrophy. Sci Adv 5, eaaw7099.
29. Lim, K. R. Q., Maruyama, R., Echigoya, Y., Nguyen, Q., Zhang, A., Khawaja, H., Sen Chandra, S., Jones, T., Jones, P., Chen, Y. W., et al. (2020). Inhibition of DUX4 expression with antisense LNA gapmers as a therapy for facioscapulohumeral muscular dystrophy. Proc Natl Acad Sci U S A 117, 16509-16515.
30. Amini Chermahini, G., Rashnonejad, A., and Harper, S.Q. (2019). RNAscope in situ hybridization-based method for detecting DUX4 RNA expression in vitro. RNA 25, 1211-1217.
31. Wallace, L. M., Saad, N. Y., Pyne, N. K., Fowler, A. M., Eidahl, J. O., Domire, J. S., Griffin, D. A., Herman, A. C., Sahenk, Z., Rodino-Klapac, L. R., et al. (2018). Pre-clinical Safety and Off-Target Studies to Support Translation of AAV-Mediated RNAi Therapy for FSHD. Mol Ther Methods Clin Dev 8, 121-130.
32. Verma, A. (2018). Recent Advances in Antisense Oligonucleotide Therapy in Genetic Neuromuscular Diseases. Ann Indian Acad Neurol 21, 3-8.
33. ldeue, T., Adachi, S., Naganuma, T., Tanigawa, A., Natsume, T., and Hirose, T. (2012). U7 small nuclear ribonucleoprotein represses histone gene transcription in cell cycle-arrested cells. Proc Natl Acad Sci U S A 109, 5693-5698.
34. Schumperli, D., and Pillai, R. S. (2004). The special Sm core structure of the U7 snRNP: far-reaching significance of a small nuclear ribonucleoprotein. Cell Mol Life Sci 61, 2560-2570.
35. Goyenvalle, A., Babbs, A., van Ommen, G. J., Garcia, L., and Davies, K. E. (2009). Enhanced exon-skipping induced by U7 snRNA carrying a splicing silencer sequence: Promising tool for DMD therapy. Mol Ther 17, 1234-1240.
36. Goyenvalle, A., Wright, J., Babbs, A., Wilkins, V., Garcia, L., and Davies, K. E. (2012). Engineering multiple U7snRNA constructs to induce single and multiexon-skipping for Duchenne muscular dystrophy. Mol Ther 20, 1212-1221.
37. Nualkaew, T., Jearawiriyapaisarn, N., Hongeng, S., Fucharoen, S., Kole, R., and Svasti, S. (2019). Restoration of correct beta(IVS2-654)-globin mRNA splicing and HbA production by engineered U7 snRNA in beta-thalassaemia/HbE erythroid cells. Sci Rep 9, 7672.
38. Biferi, M. G., Cohen-Tannoudji, M., Cappelletto, A., Giroux, B., Roda, M., Astord, S., Marais, T., Bos, C., Voit, T., Ferry, A., et al. (2017). A New AAV10-U7-Mediated Gene Therapy Prolongs Survival and Restores Function in an ALS Mouse Model. Mol Ther 25, 2038-2052.
39. Wein, N., Vulin, A., Falzarano, M. S., Szigyarto, C. A., Maiti, B., Findlay, A., Heller, K. N., Uhlen, M., Bakthavachalu, B., Messina, S., et al. (2014). Translation from a DMD exon 5 IRES results in a functional dystrophin isoform that attenuates dystrophinopathy in humans and mice. Nat Med 20, 992-1000.
40. Jones, T. I., Chen, J. C., Rahimov, F., Homma, S., Arashiro, P., Beermann, M. L., King, O. D., Miller, J. B., Kunkel, L. M., Emerson, C. P., Jr., et al. (2012). Facioscapulohumeral muscular dystrophy family studies of DUX4 expression: evidence for disease modifiers and a quantitative model of pathogenesis. Hum Mol Genet 21, 4419-4430.
41. LoRusso, S., Johnson, N. E., McDermott, M. P., Eichinger, K., Butterfield, R. J., Carraro, E., Higgs, K., Lewis, L., Mul, K., Sacconi, S., et al. (2019). Clinical trial readiness to solve barriers to drug development in FSHD (ReSolve): protocol of a large, international, multi-center prospective study. BMC Neurol 19, 224.
42. Mul, K., Vincenten, S. C. C., Voermans, N. C., Lemmers, R., van der Vliet, P. J., van der Maarel, S. M., Padberg, G. W., Horlings, C. G. C., and van Engelen, B. G. M. (2017). Adding quantitative muscle MRI to the FSHD clinical trial toolbox. Neurology 89, 2057-2065.
43. Wang, L. H., Friedman, S. D., Shaw, D., Snider, L., Wong, C. J., Budech, C. B., Poliachik, S. L., Gove, N. E., Lewis, L. M., Campbell, A. E., et al. (2019). MRI-informed muscle biopsies correlate MRI with pathology and DUX4 target gene expression in FSHD. Hum Mol Genet 28, 476-486.
44. Wong, C. J., Wang, L. H., Friedman, S. D., Shaw, D., Campbell, A. E., Budech, C. B., Lewis, L. M., Lemmers, R., Statland, J. M., van der Maarel, S. M., et al. (2020). Longitudinal measures of RNA expression and disease activity in FSHD muscle biopsies. Hum Mol Genet 29, 1030-1043.
45. Geng, L. N., Yao, Z., Snider, L., Fong, A. P., Cech, J. N., Young, J. M., van der Maarel, S. M., Ruzzo, W. L., Gentleman, R. C., Tawil, R., et al. (2012). DUX4 activates germline genes, retroelements, and immune mediators: implications for facioscapulohumeral dystrophy. Dev Cell 22, 38-51.
46. Jagannathan, S., Shadle, S. C., Resnick, R., Snider, L., Tawil, R. N., van der Maarel, S. M., Bradley, R. K., and Tapscott, S. J. (2016). Model systems of DUX4 expression recapitulate the transcriptional profile of FSHD cells. Hum Mol Genet 25, 4419-4431.
47. van den Heuvel, A., Mahfouz, A., Kloet, S. L., Balog, J., van Engelen, B. G. M., Tawil, R., Tapscott, S. J., and van der Maarel, S. M. (2019). Single-cell RNA sequencing in facioscapulohumeral muscular dystrophy disease etiology and development. Hum Mol Genet 28, 1064-1075.
48. Sharma, V., Harafuji, N., Belayew, A., and Chen, Y. W. (2013). DUX4 differentially regulates transcriptomes of human rhabdomyosarcoma and mouse C2C12 cells. PLoS One 8, e64691.
49. Rojas, L. A., Valentine, E., Accorsi, A., Maglio, J., Shen, N., Robertson, A., Kazmirski, S., Rahl, P., Tawil, R., Cadavid, D., et al. (2020). P38a Regulates Expression of DUX4 in a Model of Facioscapulohumeral Muscular Dystrophy. J Pharmacol Exp Ther. 374(3):489-498.
50. Wein, N., Vulin, A., Findlay, A. R., Gumienny, F., Huang, N., Wilton, S. D., and Flanigan, K. M. (2017). Efficient Skipping of Single Exon Duplications in DMD Patient-Derived Cell Lines Using an Antisense Oligonucleotide Approach. J Neuromuscul Dis 4, 199-207.
51. Voit, T., Topaloglu, H., Straub, V., Muntoni, F., Deconinck, N., Campion, G., De Kimpe, S. J., Eagle, M., Guglieri, M., Hood, S., et al. (2014). Safety and efficacy of drisapersen for the treatment of Duchenne muscular dystrophy (DEMAND II): an exploratory, randomised, placebo-controlled phase 2 study. Lancet Neurol 13, 987-996.
52. Kinali, M., Arechavala-Gomeza, V., Feng, L., Cirak, S., Hunt, D., Adkin, C., Guglieri, M., Ashton, E., Abbs, S., Nihoyannopoulos, P., et al. (2009). Local restoration of dystrophin expression with the morpholino oligomer AVI-4658 in Duchenne muscular dystrophy: a single-blind, placebo-controlled, dose-escalation, proof-of-concept study. Lancet Neurol 8, 918-928.
53. Mendell, J. R., Goemans, N., Lowes, L. P., Alfano, L. N., Berry, K., Shao, J., Kaye, E. M., Mercuri, E., Eteplirsen Study, G., and Telethon Foundation, D.M.D.I.N. (2016). Longitudinal effect of eteplirsen versus historical control on ambulation in Duchenne muscular dystrophy. Ann Neurol 79, 257-271.
54. Carrell, S. T., Carrell, E. M., Auerbach, D., Pandey, S. K., Bennett, C. F., Dirksen, R. T., and Thornton, C.A. (2016). Dmpk gene deletion or antisense knockdown does not compromise cardiac or skeletal muscle function in mice. Hum Mol Genet 25, 4328-4338.
55. Aoki, Y., Nagata, T., Yokota, T., Nakamura, A., Wood, M. J., Partridge, T., and Takeda, S. (2013). Highly efficient in vivo delivery of PMO into regenerating myotubes and rescue in laminin-alpha2 chain-null congenital muscular dystrophy mice. Hum Mol Genet 22, 4914-4928.
56. Heemskerk, H., de Winter, C., van Kuik, P., Heuvelmans, N., Sabatelli, P., Rimessi, P., Braghetta, P., van Ommen, G. J., de Kimpe, S., Ferlini, A., et al. (2010). Preclinical PK and PD studies on 2′-O-methyl-phosphorothioate RNA antisense oligonucleotides in the mdx mouse model. Mol Ther 18, 1210-1217.
57. Shimizu-Motohashi, Y., Komaki, H., Motohashi, N., Takeda, S., Yokota, T., and Aoki, Y. (2019). Restoring Dystrophin Expression in Duchenne Muscular Dystrophy: Current Status of Therapeutic Approaches. J Pers Med 9.
58. Gonzalez-Barriga, A., Kranzen, J., Croes, H. J., Bijl, S., van den Broek, W. J., van Kessel, I. D., van Engelen, B. G., van Deutekom, J. C., Wieringa, B., Mulders, S. A., et al. (2015). Cell membrane integrity in myotonic dystrophy type 1: implications for therapy. PLoS One 10, e0121556.
59. Brun, C., Suter, D., Pauli, C., Dunant, P., Lochmuller, H., Burgunder, J. M., Schumperli, D., and Weis, J. (2003). U7 snRNAs induce correction of mutated dystrophin pre-mRNA by exon skipping. Cell Mol Life Sci 60, 557-566.
60. Stadler, G., Rahimov, F., King, O. D., Chen, J. C., Robin, J. D., Wagner, K. R., Shay, J. W., Emerson, C. P., Jr., and Wright, W. E. (2013). Telomere position effect regulates DUX4 in human facioscapulohumeral muscular dystrophy. Nat Struct Mol Biol 20, 671-678.
61. Liu, J., and Gall, J. G. (2007). U bodies are cytoplasmic structures that contain uridine-rich small nuclear ribonucleoproteins and associate with P bodies. PNAS 104(28), 11655-11659.

The disclosure has been described in terms of particular embodiments found or proposed to comprise specific modes for the practice of the disclosure. Various modifications and variations of the disclosure will be apparent to those skilled in the art without departing from the scope and spirit of the disclosure. Although the disclosure has been described in connection with specific embodiments, it should be understood that the methods of the disclosure as claimed should not be unduly limited to such specific embodiments. Indeed, various modifications of the described modes for carrying out the methods that are obvious to those skilled in the relevant fields are intended to be within the scope of the following claims.

We claim:

1. A nucleic acid encoding a U7 double homeobox 4 (DUX4) antisense ribonucleic acid (asRNA), the nucleic acid comprising
(a) a nucleotide sequence comprising at least 90% identity to the sequence set forth in any one of SEQ ID NOs: 1-18;
(b) the nucleotide sequence set forth in any one of SEQ ID NOs: 1-18; or
(c) a combination of the nucleotide sequences of (a) and/or (b).

2. A nucleic acid comprising a nucleotide sequence encoding a U7 double homeobox 4 (DUX4) antisense sequence that specifically hybridizes to a DUX4 target nucleotide sequence set forth in any one of SEQ ID NOs: 19-36 or a combination of the nucleotide sequences.

3. The nucleic acid of claim 1 or 2 further comprising a promoter nucleotide sequence.

4. The nucleic acid of claim 3, wherein the promoter is any of a U6 promoter, a U7 promoter, a tRNA promoter, a H1 promoter, a minimal CMV promoter, a T7 promoter, an EF1-alpha promoter, a Minimal EF1-alpha promoter, or a muscle-specific promoter.

5. The nucleic acid of claim 4, wherein the muscle-specific promoter is a unc45b promoter, a tMCK promoter, a minimal MCK promoter, a CK6 promoter, a CK7 promoter, a MHCK7 promoter, or a CK1 promoter.

6. A nanoparticle, extracellular vesicle, exosome, or vector comprising the nucleic acid of any one of claims 1-5 or a combination of any one or more thereof.

7. The vector of claim 6, wherein the vector is a viral vector.

8. The viral vector of claim 7, wherein the viral vector is an adeno-associated virus (AAV), adenovirus, lentivirus, retrovirus, poxvirus, baculovirus, herpes simplex virus, vaccinia virus, or a synthetic virus.

9. The viral vector of claim 7 or 8, wherein the viral vector is an AAV.

10. The viral vector of claim 9, wherein the AAV lacks rep and cap genes.

11. The viral vector of claim 9 or 10, wherein the AAV is a recombinant AAV (rAAV) or a self-complementary recombinant AAV (scAAV).

12. The viral vector of any one of claims 9-11, wherein the AAV is rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh.10, or rAAV-B1.

13. The viral vector of any one of claims 9-12, wherein the AAV is rAAV-9.

14. A composition comprising
(a) the nucleic acid of any one of claims 1-5;
(b) the nanoparticle, extracellular vesicle, exosome, or vector of claim 6; or
(c) the viral vector of any one of claims 7-13; and
a pharmaceutically acceptable carrier.

15. A method of inhibiting and/or interfering with expression of a double homeobox 4 (DUX4) gene in a cell comprising contacting the cell with
(a) the nucleic acid of any one of claims 1-5;
(b) the nanoparticle, extracellular vesicle, exosome, or vector of claim 6;
(c) the viral vector of any one of claims 7-13; or
(d) the composition of claim 14.

16. A method of treating a subject having a muscular dystrophy comprising administering to the subject an effective amount of
(a) the nucleic acid of any one of claims 1-5;
(b) the nanoparticle, extracellular vesicle, exosome, or vector of claim 6;
(c) the viral vector of any one of claims 7-13; or
(d) the composition of claim 14.

17. The method of claim 16, wherein the muscular dystrophy is facioscapulohumeral muscular dystrophy (FSHD).

18. A method of treating a subject having a cancer comprising administering to the subject an effective amount of
(a) the nucleic acid of any one of claims 1-5;
(b) the nanoparticle, extracellular vesicle, exosome, or vector of claim 6;
(c) the viral vector of any one of claims 7-13; or
(d) the composition of claim 14.

19. The method of claim 18, wherein the cancer is a sarcoma.

20. Use of
(a) the nucleic acid of any one of claims 1-5;
(b) the nanoparticle, extracellular vesicle, exosome, or vector of claim 6;
(c) the viral vector of any one of claims 7-13; or
(d) the composition of claim 14
for the preparation of a medicament for inhibiting expression of a double homeobox 4 (DUX4) gene in a cell.

21. Use of
(a) the nucleic acid of any one of claims 1-5;
(b) the nanoparticle, extracellular vesicle, exosome, or vector of claim 6;
(c) the viral vector of any one of claims 7-13; or (d) the composition of claim 14
for inhibiting expression of a double homeobox 4 (DUX4) gene in a cell.

22. Use of
(a) the nucleic acid of any one of claims 1-5;
(b) the nanoparticle, extracellular vesicle, exosome, or vector of claim 6;
(c) the viral vector of any one of claims 7-13; or
(d) the composition of claim 14
for the preparation of a medicament for treating or ameliorating a muscular dystrophy.

23. Use of
(a) the nucleic acid of any one of claims 1-5;
(b) the nanoparticle, extracellular vesicle, exosome, or vector of claim 6;
(c) the viral vector of any one of claims 7-13; or
(d) the composition of claim 14
for treating or ameliorating a muscular dystrophy.

24. The use of claim 22 or 23, wherein the muscular dystrophy is facioscapulohumeral muscular dystrophy.

25. Use of
(a) the nucleic acid of any one of claims 1-5;
(b) the nanoparticle, extracellular vesicle, exosome, or vector of claim 6;
(c) the viral vector of any one of claims 7-13; or
(d) the composition of claim 14
for the preparation of a medicament for treating or ameliorating a cancer.

26. Use of
(a) the nucleic acid of any one of claims 1-5;
(b) the nanoparticle, extracellular vesicle, exosome, or vector of claim 6;
(c) the viral vector of any one of claims 7-13; or
(d) the composition of claim 14
for treating or ameliorating a cancer.

27. The use of claim 25 or 26, wherein the cancer is a sarcoma.

28. The
(a) nucleic acid of any one of claims 1-5;
(b) nanoparticle, extracellular vesicle, exosome, or vector of claim 6;
(c) viral vector of any one of claims 7-13;
(d) composition of claim 14;
(e) method of any one of claims 15-19; or
(e) use of any one of claims 20-27,
wherein the nucleic acid, nanoparticle, extracellular vesicle, exosome, vector, composition, or medicament is formulated for intramuscular injection, transdermal transport or injection into the blood stream.
